(19) United States
(12) Patent Application Publication
(10) Pub. No.: US 2009/0215666 A1
(43) Pub. Date: Aug. 27, 2009

(54) VIVIT POLYPEPTIDES, THERAPEUTIC AGENT COMPRISING THE SAME, AND METHOD OF SCREENING FOR ANTI-CANCER AGENT

(75) Inventors: Yusuke Nakamura, Tokyo (JP); Toyomasa Katagiri, Tokyo (JP); Koichiro Inaki, Kanagawa (JP)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Oncotherapy Science, Inc., Kanagawa (JP); The University of Tokyo, Bunkyo-ku (JP)

(21) Appl. No.: 11/912,866

(22) PCT Filed: Jul. 14, 2006
(86) PCT No.: PCT/JP2006/314442

§ 371 Date: Nov. 6, 2008

Related U.S. Application Data
(60) Provisional application No. 60/703,791, filed on Jul. 28, 2005.

(51) Int. Cl. A61K 38/02; C07K 2/00; C07K 7/06; C07K 7/08; C07K 14/00; G01N 33/53; C12N 5/00; C07H 21/00; A61K 31/7052; A61P 35/00

(52) U.S. Cl. 514/2; 530/300; 530/330; 530/329; 530/328; 530/327; 530/326; 530/325; 530/324; 436/501; 435/7.2; 435/375; 536/23.1; 514/44 R

(57) ABSTRACT

The present invention provides polypeptides useful for treating and preventing cancer. The present invention also provides therapeutic agents or methods for treating cancer using the polypeptides. The polypeptides of the present invention are composed of an amino acid sequence which comprises VIVIT and is preferably a polypeptide in which the motif sequence PxIxIT at positions 37 to 41 of the amino acid sequence of the C1958 protein (SEQ ID NO: 2) is replaced with PVIVIT. The polypeptides of the present invention can be introduced into cancer cells by modifying the polypeptides with transfection agents such as poly-arginine. The present invention provides methods and kits for identifying inhibitors of the interaction between C1958 and PPP3CA which find utility in the treatment and prevention of cancer. Also disclosed herein are compositions for treating or preventing cancer identified by the screening method of the present invention and methods of using same in the treatment and prevention of cancer.

This application claims the benefit of U.S. Provisional Application Ser. No. 60/703,791 filed Jul. 28, 2005, the contents of which are hereby incorporated by reference in their entirety.

### TECHNICAL FIELD

The present invention relates to the field of biological science, more specifically to the field of cancer research. More particularly, the present method relates to the discovery that C1958 interacts with calcineurin.

### BACKGROUND ART

Pancreatic ductal adenocarcinoma (PDACa) is the fifth leading cause of cancer death in the western world and has one of the highest mortality rates of any malignancy, with a 5-year survival rate of only 4%. In the USA, each year, an estimated 30,700 patients are diagnosed with pancreatic cancer and nearly 30,000 die of these diseases. The vast majority of patients are diagnosed at an advanced stage of disease. At this point, current therapies are generally ineffective and life expectancy is just a few months. Only surgical resection can offer the possibility of cure, but only 10-20% of patients with PDACa can undergo potentially curative resection. Even after curative surgery, 80-90% of the patients relapse and die of the disease. Some improvements in surgical outcome or quality of life occur in patients who also receive chemotherapy including gemcitabine and/or radiation, although the impact on long-term survival has been minimal due to the intense resistance of PDACa to any treatment. At this point, management of most patients focuses on palliation.

Therefore, establishment of a novel molecular therapy for PDACa and identification of novel therapeutic molecular targets for PDACa are urgent issues for pancreatic cancer treatment now.

The present inventors previously analyzed gene-expression profiles of cancer cells from 18 pancreatic cancer patients using a cDNA microarray representing 23,040 human genes, and identified 265 genes that were commonly up-regulated in pancreatic cancer cells (Nakamura T, (2004) Oncogene. 23, 2385-400). This analysis revealed that C1958V1 and C1958V2 were up-regulated in pancreatic cancer specifically. Results of semi-quantitative RT-PCR analysis also showed elevated expression in 11 of 12 pancreatic cancer patients, and 4 of 5 pancreatic cancer cell lines compared with normal pancreatic duct cells. Furthermore, C1958-specific siRNA significantly suppressed the growth of pancreatic cancer cells (WO2004/31411).

### SUMMARY OF THE INVENTION

An objective of the present invention is to provide compounds useful in the treatment and/or prevention of cancer. Alternatively, an objective of the present invention is to provide pharmaceutical compositions and methods for of the treatment and/or prevention of cancer using the compounds.

The present inventors have proved that suppression of C1958V1 and C1958V2 expressions can achieve the inhibition of cancer proliferation. It has been found that C1958 has three splicing variants. These variants are named C1958V1, C1958V2, and C1958V3 respectively. cDNA of C1958V1 (SEQ ID NO: 1, 881 nucleotides) encodes the amino acid sequence set forth in SEQ ID NO: 2, and cDNA of C1958V2 (SEQ ID NO: 3, 893 nucleotides) encodes the amino acid sequence set forth in SEQ ID NO: 4. cDNA of C1958V3 consists of 1503 nucleotides.

When the cDNA of C1958 is used as a probe in Northern blot analyses, two transcripts of about 1.7 kb and 1.1 kb is detected The 1.7 kb transcript is highly expressed in lymph nodes, and slightly expressed in stomach, trachea, and bone marrow. It has been observed that the 1.1 kb transcript is expressed in placenta, and that it is expressed at extremely low level in liver, thyroid gland, trachea, and bone marrow. It has been also confirmed that the expressions of C1958V1 and 1958V2 are specifically elevated in pancreatic cancer cell lines.

Furthermore, when the endogenous C1958 expression in a pancreatic cancer cell line is inhibited by siRNA specific to C1958, the cell proliferation is suppressed (WO2004/31411). This result indicates that C1958 is a necessary molecule for proliferation or survival of cancer cells. These results show that control of cancer cell proliferation can be achieved by controlling C1958 function. Therefore, the present inventors first searched for a molecule that binds to the C1958 protein. As a result, calcineurin A subunit PP2B (hereinafter referred to as PPP3CA) was identified as a molecule binding to the C1958 protein. PPP3CA was also found to bind to the phosphorylated C1958 protein.

It is known that PPP3CA binds to the nuclear factor of activated T-cells (FAM). Interaction of both molecules is considered to be an important mechanism in T cell proliferation. It has been reported that PPP3CA interacts with NFAT at the conserved specific motif PxIxIT (Kiani A. et al., Immunity 2000; 12: 359-72). In fact, it was also observed that a synthetic peptide containing this motif effectively inhibits the interaction between PPP3CA and NFAT (Aramburu J. et al., Science 1999: 285, 2129-33). The specific motif PxIxIT is conserved at positions from 36 to 41 (PDIIIT) of the amino acid sequence of C1958V1 protein (SEQ ID NO: 2). Therefore, the present inventors confirmed that C1958 interacts with PPP3CA at this motif. Specifically, a C1958 variant without this motif (ΔPDIIIT-C1958) no longer binds to PPP3CA (FIG. 3A). This result indicates that the amino acids from positions 36 to 41 (PDIIIT) of SEQ ID NO: 2 are essential for binding to PPP3CA.

On the other hand, it has been recently revealed that the binding between NFAT and PPP3CA via the motif PxIxIT is strongly competitively inhibited by a peptide containing the amino acid sequence VIVIT (Val Ile Val Ile Thr/SEQ ID NO: 27) (Aramburu, J. et al., Science 1999: 285, 2129-33). Therefore, an oligopeptide containing an amino acid sequence in which the motif PxIxIT in the amino acid sequence of C1958 protein is replaced with VIVIT was prepared to examine its effect on cells. As a result, the present inventors proved that a peptide containing an amino acid sequence in which the amino acid sequence DIIIT in C1958 peptide is replaced with VIVIT shows a potent cell proliferation-inhibiting activity, thereby completing the present invention. Specifically, the present invention provides polypeptides that contain a subsequence containing Val Ile Val Ile Thr/SEQ ID NO: 27. In some preferred embodiments, the amino acid sequence Asp Ile Ile Ile Thr at positions 37 to 41 of the amino acid sequence set forth in SEQ ID NO: 2 (C1958) is replaced with Val Ile Val Ile Thr/SEQ ID NO: 27. The present invention further provides pharmaceuticals or methods using these polypeptides for prevention and/or treatment of cancer.

The present invention also relates to methods for treatment and/or prevention of cancer comprising the step of administering a polypeptide that contains Val Be Val Ile Thr/SEQ ID NO: 27, for example a polypeptide having at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27; or a polynucleotide encoding the same. Furthermore, the present invention relates to the use of polypeptides of the invention; or the use of nucleotides encoding the same, in manufacturing pharmaceutical formulations for the treatment and/or prevention of cancer.

The present invention relates to polypeptides that inhibit cell proliferation of cancers. The present invention is also based on the finding that C1958 and calcineurin interact in vivo. In view of that discovery and that the expression of C1958 is associated with pancreatic cancer (see, e.g., PCT Publication No. WO2004/31411), the present invention provides methods of screening for compounds to treat cancer by identifying compounds that inhibit the binding of C1958 and calcineurin.

Accordingly, it is an objective of the present invention is to provide methods of screening for compounds useful in treating and preventing cancer. In one embodiment, the method of the present invention comprises the steps of:
(a) contacting a polypeptide comprising a PPP3 CA-binding domain (i.e., a domain comprising the specific motif, PxIxIT) of a C1958 polypeptide with a polypeptide comprising a C1958-binding domain of a PPP3CA polypeptide in the presence of a test compound;
(b) detecting binding between the polypeptides; and

(c) selecting a test compound that inhibits binding between the polypeptides.

The present invention also provides kits for screening for a compound useful in treating or preventing cancer. In some embodiments, the kit comprises:

(a) a first polypeptide comprising a PPP3CA-binding domain of a C1958 polypeptide;
(b) a second polypeptide comprising a C1958-binding domain of a PPP3CA polypeptide, and

(c) a reagent that detects the interaction between the first and second polypeptides.

In some embodiments, the first polypeptide, i.e., the polypeptide comprising the PPP3CA-binding domain comprises a C1958 polypeptide. In some embodiments, the polypeptide comprising the PPP3CA-binding domain, e.g. C1958 polypeptide may be phosphorylated form. Alternatively, in some embodiments, the polypeptide comprising the PPP3CA-binding domain is a polypeptide comprising amino acid sequence from positions 36 to 41 of the amino acid sequence of SEQ ID NO: 2. Likewise, in some embodiments, the second polypeptide, i.e., the polypeptide comprising the C1958-binding domain, comprises a PPP3 CA polypeptide.

In some embodiments, the polypeptide comprising the PPP3CA-binding domain is expressed in a living cell.

In some embodiments, the reagent that detects the interaction between the first and second polypeptides comprises a reagent that detects e.g. an association between the polypeptide comprising the PPP3CA-binding domain and the polypeptide comprising the C1958 binding domain.

The present invention also provides methods for treating or preventing cancers in a subject. In some embodiments, the method comprises the step of administering a pharmaceutically effective amount of a compound that inhibits binding between a C1958 polypeptide and a PPP3CA polypeptide.

The present invention also provides compositions for treating or preventing cancers. In some embodiments, the composition comprises a pharmaceutically acceptable carrier and a pharmaceutically effective amount of a compound selected by the method the steps of:

(a) contacting a polypeptide comprising a PPP3CA-binding domain of a C1958 polypeptide with a polypeptide comprising a C1958-binding domain of a PPP3CA polypeptide in the presence of a test compound;

(b) detecting binding between the polypeptides; and

(c) selecting a test compound that inhibits binding between the polypeptides.

In some embodiments, the composition comprises a pharmaceutically effective amount of a compound that inhibits the binding between a C1958 polypeptide and a PPP3CA polypeptide, and a pharmaceutically acceptable carrier.

These and other objects and features of the invention will become more fully apparent when the following detailed description is read in conjunction with the accompanying figures and examples. However, it is to be understood that both the foregoing summary of the invention and the following detailed description are of a preferred embodiment, and not restrictive of the invention or other alternate embodiments of the invention.

### BRIEF DESCRIPTION OF THE DRAWINGS

FIG. 1 shows the results of immunocytochemical analysis (a, b) and Immunohistochemical analysis (c-g) for C1958 protein. (a, b) Immunocytochemical analysis for C1958 protein using PK-1 and KLM-1 pancreatic cancer cells. Staining by rabbit polyclonal antibody, raised against full-length C1958 recombinant protein, (green) demonstrates plasma-membrane localization of C1958. Blue, DAPI. (c-g) Immunohistochemical analysis for C1958 protein using pancreatic cancer and normal human tissue sections. (c) pancreatic cancer (d) kidney, (e) Liver, (f) Heart, (g) Lung. Brown; C1958, blue; hematoxylin counter-staining.

FIG. 2 shows the result of Western blot analysis for exogenous C1958 in various cell lines.

Western blot analysis for exogenous C1958 in Cos-7 cell (a) and endogenous C1958 in pancreatic cancer cell lines (b). Upper and lower arrows indicate the phosphorylated and non-phosphorylated form of C1958, respectively. ACTB: b-actin used as an internal control.

FIG. 3 shows the result of Immunoprecipitation assay. Immunoprecipitation assay demonstrating the interaction between C1958 and PPP3CA. Flag-tagged C1958, ΔPDIIIT mutant, and HA-tagged PPP3CA were exogenously expressed in Cos-7 cells.

PPP3CA/C1958 (mutant) complex was immunoprecipitated by anti-41A antibody and immunoblotted with anti-Flag antibody. Upper and lower arrows indicate the phosphorylated and non-phosphorylated form of C1958, respectively.

FIG. 4 depicts the anti-cell growth effect of inhibitory peptide comprising PXIXIT motif. Anti-cell growth activity of cell-permeable inhibitory peptide flanking the binding site, PXIXIT motif, of C1958 to PPP3CA. PK-1 cells were treated with the peptides and MTT assays were performed at indicated days. Amino acid sequences of the peptides are shown in Table 1.

FIG. 5 depicts the C1958-independent anti-cell growth activity of C1958VIVIT peptide. —C1958 negative or weakly expressing Panc-1, NHDF, and HEK293T cells were incubated with the peptides and the cell viability was quantified by MTT assay, similarly as in FIG. 4.

FIG. 6 depicts the In vivo anti-tumor growth activity of C1958VIVIT peptide. The peptides were injected intravenously (upper) or intratumorally (lower) to subcutaneous xenografts (PK-1 cells) tumor in mice for 21 consecutive days. Tumor volumes are shown as percentages of that at day 0.

FIG. 7. Flow cytometric analysis for C1958VIVIT-treated cells. PK-1 cells were incubated without (−) or with negative control (Cont.) at 40 μM or with C1958-VIVIT (CV) at 10, 20, and 40 μM for 12 hr. After the incubation, the number of cells in sub-G1 fraction was counted with FACS calibur and shown as a percentage of whole cells in all fractions.

### DETAILED DESCRIPTION OF THE PREFERRED EMBODIMENTS

### I. Definitions

The words “a”, “an”, and “the” as used herein mean “at least one” unless otherwise specifically indicated.

In the context of the present invention, a “C1958 polypeptide” refers to a polypeptide whose expression is linked to pancreatic cancer. See, e.g., PCT Pub. No. WO2004/31411, incorporated by reference herein in its entirety. Exemplary C1958 polypeptides may be substantially identical to, e.g. SEQ ID NO: 2 (encoded by SEQ ID NO: 1), and Genbank accession number AB115764. The amino acid sequence of SEQ ID NO: 2 is disclosed as C1958V1 in the PCT Pub. No. WO2004131411.

In the context of the present invention, “inhibition of binding” between two proteins refers to at least reducing binding and sometimes completely preventing binding between the proteins. In some cases, the percentage of binding pairs in a sample will be decreased as compared to an appropriate (e.g., not treated with test compound, or from a non-cancer sample, or from a cancer sample) control. The reduction in the amount of proteins bound may be, e.g., less than 90%, 80%, 70%, 60%, 50%, 40%, 25%, 10%, 5%, 1% or less, than the pairs bound in a control sample.

The term “test compound” refers to any (e.g., chemically or recombinantly-produced) molecule that may disrupt protein-protein interaction between C1958 and PPP3 CA, as discussed in detail herein. In some embodiments, the test compounds have a molecular weight of less than 1,500 daltons, and in some cases less than 1,000, 800, 600, 500, or 400 daltons.

A “pharmaceutically effective amount” of a compound is a quantity that is sufficient to treat and/or ameliorate a C1958-mediated disease in an individual. An example of a pharmaceutically effective amount may an amount needed to decrease interaction between C1958 and PPP3CA when administered to an animal, so as to thereby reduce or prevent cancers. The decrease in interaction may be, e.g., at least about a 5%, 10%, 20%, 30%, 40%, 50%, 75%, 80%, 90%, 95%, 99%, or 100% change in binding.

The phrase “pharmaceutically acceptable carrier” refers to an inert substance used as a diluent or vehicle for a drug.

In the present invention, the term “functionally equivalent” means that the subject polypeptide has a biological activity of a reference polypeptide. For example, a functional equivalent of C1958 polypeptide would have the binding activity with PPP3CA like wild type C1958.

The terms “isolated” and “biologically pure” refer to material which is substantially or essentially free from components which normally accompany it as found in its native state. However, the term “isolated” is not intended to refer to the components present in an electrophoretic gel or other separation medium. An isolated component is free from such separation media and in a form ready for use in another application or already in use in the new application/milieu.

The phrase “conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicitly described in each disclosed sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” wherein the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (1), Leucine (L), Methionine (M), Valine (V);

6) Phenylalanine (F), Tyrosine (Y), Tryptophan (W);

7) Serine (S), Threonine (T); and

8) Cysteine (C), Methionine (M) (see, e.g., Creighton, Proteins (1984)).

In the context of the present invention, a “percentage of sequence identity” is determined by comparing two optimally aligned sequences over a comparison window, wherein the portion of the polynucleotide sequence in the comparison window may comprise additions or deletions (i.e., gaps) as compared to the reference sequence (e.g., a polypeptide of the invention), which does not comprise additions or deletions, for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same sequences. Two sequences are “substantially identical” if two sequences have a specified percentage of amino acid residues or nucleotides that are the same (i.e., 60% identity, optionally 65%, 70%, 75%, 80%, 85%, 90%, or 95% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides in length, or more preferably over a region that is 100 to 500 or 1000 or more nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1981) Adv. Appl. Math. 2:482-489, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

Two examples of algorithms that are suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment The BLASTN program (for nucleotide sequences) uses as defaults a word length (W) of 11, an expectation (E) of 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-7). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “small organic molecules” refers to molecules of a size comparable to those organic molecules generally used in pharmaceuticals. The term excludes biological macromolecules (e.g., proteins, nucleic acids, etc.). Preferred small organic molecules range in size up to about 5000 Da, e.g., up to 2000 Da, or up to about 1000 Da The terms “label” and “detectable label” are used herein to refer to any composition detectable by spectroscopic, photochemical, biochemical, immunochemical, electrical, optical or chemical means. Such labels include biotin for staining with labeled streptavidin conjugate, magnetic beads (e.g., DYNABEADS™), fluorescent dyes (e.g., fluorescein, Texas red, rhodamine, green fluorescent protein, and the like), radiolabels (e.g. ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241. Means of detecting such labels are well known to those of skill in the art. Thus, for example, radiolabels may be detected using photographic film or scintillation counters, fluorescent markers may be detected using a photodetector to detect emitted light Enzymatic labels are typically detected by providing the enzyme with a substrate and detecting, the reaction product produced by the action of the enzyme on the substrate, and calorimetric labels are detected by simply visualizing the colored label.

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, Antibodies: a laboratory manual, Cold Spring Harbor, N.Y., 1988; Hilyard et al., Protein Engineering: A practical approach (IRL Press 1992); Bonrabeck, Antibody Engineering, 2d ed. (Oxford University Press 1.995); each of which is incorporated herein by reference).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al. (1993) Proc Natl Acad Sci U S A. 90:6444, Gruber et al. (1994) J Immunol: 5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional spaces.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” refer to the variable region of an immunoglobulin heavy chain of an antibody, including the heavy chain of an Fv, scFv, or Fab. References to “V_{L}” refer to the variable region of an immunoglobulin light chain, including the light chain of an Fv, scFv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-327 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

The terms “epitope” and “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operable linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

Unless otherwise defined, all technical and scientific terms used herein have the same meaning as commonly understood by one of ordinary skill in the art to which this invention belongs. In case of conflict, the present specification, including definitions, will control.

### II VIVIT Polypeptide

The present invention relates to polypeptides that contain Val Ile Val Be Thr/SEQ ID NO: 27. In some preferred embodiments, the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27. The amino acid sequence set forth in SEQ ID NO: 2 is disclosed in WO2004/31411. It has been known that cancer cell proliferation can be controlled by inhibiting the expression of the amino acid sequence. However, it is a novel finding proved by the present inventors that a fragment containing a sequence with a specific mutation in the above amino acid sequence inhibits the cancer cell proliferation.

The polypeptides of the present invention include those meeting either of the following two conditions A and B. Hereinafter, an amino acid sequence of polypeptide meeting either of the following two conditions A and B may be referred to as “a polypeptide comprising the selected amino acid sequence.”

A: Containing an amino acid sequence in which Asp Ile le Ile Thr at positions 37 to 41 of the amino acid sequence set forth in SEQ ID NO: 2 (C1958) is replaced with Val Ile Val Ile Thr/SEQ ID NO: 27 (VIVIT), and

B: Containing the amino acid sequence Val Ile Val Ile Thr/SEQ ID NO: 27.

The polypeptides comprising the selected amino acid sequence of the present invention, can be of any length, so long as the polypeptide inhibits cancer cell proliferation. Specifically, the length of the amino acid sequence may range from 5 to 70 residues, for example, from 5 to 50, preferably from 5 to 30, more specifically from 5 to 20, further more specifically from 5 to 16 residues. For example, the amino acid sequence KHLDVPVIVITPPTPT (SEQ ID NO: 26) is preferable as the above-described selected amino acid sequence. Therefore, a polypeptide comprising or consisting of the amino acid sequence KHLDVPVIVITPPTPT (SEQ ID NO: 26) is a preferred example of the polypeptides in the present invention. The polypeptides of the present invention, which are characterized by containing the amino acid sequence VIVIT, may also be referred to as “VIVIT polypeptides.”

The polypeptides of the present invention may contain two or more “selected amino acid sequences.” The two or more “selected amino acid sequences” may be the same or different amino acid sequences. Furthermore, the “selected amino acid sequences” can be linked directly. Alternatively, they may be disposed with any intervening sequences among them.

Furthermore, the present invention relates to polypeptides homologous to the VIVIT polypeptide specifically disclosed here. In the present invention, polypeptides homologous to the VIVIT polypeptide are those which contain any mutations selected from addition, deletion, substitution and insertion of one or several amino acid residues and are functionally equivalent to the VIVIT polypeptide. The phrase “functionally equivalent to the VIVIT polypeptide” refers to having the function to inhibit the binding of C1958 to PPP3CA. The VIVIT sequence is preferably conserved in the amino acid sequences constituting polypeptides functionally equivalent to VIVIT polypeptide. Therefore, polypeptides functionally equivalent to the VIVIT peptide in the present invention preferably have amino acid mutations in sites other than the VIVIT sequence. Amino acid sequences of polypeptides functionally equivalent to the VIVIT peptide in the present invention conserve the VIVIT sequence, and have 60% or higher, usually 70% or higher, preferably 80% or higher, more preferably 90% or higher, or 95% or higher, and further more preferably 98% or higher homology to a “selected amino acid sequence”. Amino acid sequence homology can be determined using algorithms well known in the art.

Alternatively, the number of amino acids that may be mutated is not particularly restricted, so long as the VIVIT peptide activity is maintained. Generally, up to about 50 amino acids may be mutated, preferably up to about 30 amino acids, more preferably up to about 10 amino acids, and even more preferably up to about 3 amino acids. Likewise, the site of mutation is not particularly restricted, so long as the mutation does not result in the disruption of the VIVIT peptide activity.

In a preferred embodiment, the activity of the VIVIT peptide comprises apoptosis inducing effect in a C1958 expressing cell, i.e. pancreatic cancer cell. Apoptosis means cell death caused by the cell itself and is sometimes referred to as programmed cell death. Aggregation of nuclear chromosome, fragmentation of nucleus, or condensation of cytoplasm is observed in a cell undergoing apoptosis. Methods for detecting apoptosis are well known. For instance, apoptosis may be confirmed by TUNEL staining (Terminal deoxynucleotidyl Transferase Biotin-dUTP Nick End Labeling; Gavriela, Y., et al., J. Cell Biol. 119: 493-501, 1992 Mori, C., et al., Anat & Embryol. 190: 21-28, 1994.). Alternatively, DNA ladder assays, Annexin V staining, caspase assay, electron microscopy, or observation of conformational alterations on nucleus or cell membrane may be used for detecting apoptosis. Any commercially available kits may be used for detecting these behaviors in cells which are induced by apoptosis. For example, such apoptosis detection kits may be commercially available from the following providers:

LabChem. Inc.,

Promega,

BD Biosciences Pharmingen,

Calbiochem,

Takara Bio Company (CLONTECH Inc.),

CHEMICON International, Inc,

Medical & Biological Laboratories Co., Ltd. etc.

The polypeptides of the present invention can be chemically synthesized from any position based on selected amino acid sequences. Methods used in the ordinary peptide chemistry can be used for the method of synthesizing polypeptides. Specifically, the methods include those described in the following documents and Japanese Patent publications:

Peptide Synthesis, Interscience, New York, 1966; The Proteins, Vol. 2, Academic Press Inc., New York, 1976;

Peputido gousei (Peptide Synthesis), Maruzen (Inc.), 1975;

Peputido gousei no kiso to jikken (Fundamental and Experimental Peptide Synthesis), Maruzen (Inc.), 1985;

Iyakuhin no kaihatsu (Development of Drug), Sequel, Vol. 14: Peputido gousei (Peptide Synthesis), Hirokawa Shoten, 1991;

International Patent Publication WO99/67288.

The polypeptides of the present invention can be also synthesized by known genetic engineering techniques. An example of genetic engineering techniques is as follows. Specifically, DNA encoding a desired peptide is introduced into an appropriate host cell to prepare a transformed cell. The polypeptides of the present invention can be obtained by recovering polypeptides produced by this transformed cell. Alternatively, a desired polypeptide can be synthesized with an in vitro translation system, in which necessary elements for protein synthesis are reconstituted in vitro.

When genetic engineering techniques are used, the polypeptide of the present invention can be expressed as a fused protein with a peptide having a different amino acid sequence. A vector expressing a desired fusion protein can be obtained by linking a polynucleotide encoding the polypeptide of the present invention to a polynucleotide encoding a different peptide so that they are in the same reading frame, and then introducing the resulting nucleotide into an expression vector. The fusion protein is expressed by transforming an appropriate host with the resulting vector. Different peptides to be used in forming fusion proteins include the following peptides:

FLAG (Hopp, T. P. et al., BioTechnology (1988) 6, 1204-1210),

6×His consisting of six His (histidine) residues, 10×His,

Influenza hemagglutinin (HA),

Human c-myc fragment,

VSV-GP fragment,

p18 HIV fragment,

T7-tag,

HSV-tag,

E-tag,

SV40T antigen fragment,

Ick tag,

α-tubulin fragment,

B-tag,

Protein C fragment,

GST (glutathione-S-transferase),

HA (Influenza hemagglutinin),

Immunoglobulin constant region,

β-galactosidase, and

MBP (maltose-binding protein).

The polypeptide of the present invention can be obtained by treating the fusion protein thus produced with an appropriate protease, and then recovering the desired polypeptide. To purify the polypeptide, the fusion protein is captured in advance with affinity chromatography that binds with the fusion protein, and then the captured fusion protein can be treated with a protease. With the protease treatment, the desired polypeptide is separated from affinity chromatography, and the desired polypeptide with high purity is recovered.

The polypeptides of the present invention include modified polypeptides which meet either of the aforementioned conditions A and B. In the present invention, the term “modified” refers, for example, to binding with other substances. The other substances include organic compounds such as peptides, lipids, saccharides, and various naturally-occurring or synthetic polymers. The polypeptides of the present invention may have any modifications so long as the polypeptides retain the desired activity of inhibiting the binding of C1959 to PPP3CA. Modifications can also confer additive functions on the polypeptides of the invention. Examples of the additive functions include targetability, deliverability, and stabilization.

Preferred examples of modifications in the present invention include, for example, the introduction of a cell-membrane permeable substance. Usually, the intracellular structure is cut off from the outside by the cell membrane. Therefore, it is difficult to efficiently introduce an extracellular substance into cells. Cell membrane permeability can be conferred on the polypeptides of the present invention by modifying the polypeptides with a cell-membrane permeable substance. As a result, by contacting the polypeptide of the present invention with a cell, the polypeptide can be delivered into the cell to act thereon.

The “cell-membrane permeable substance” refers to a substance capable of penetrating the mammalian cell membrane to enter the cytoplasm. For example, a certain liposome fuses with the cell membrane to release the content into the cell. Meanwhile, a certain type of polypeptide penetrates the cytoplasmic membrane of mammalian cell to enter the inside of the cell. For polypeptides having such a cell-entering activity, cytoplasmic membranes and such in the present invention are preferable as the substance. Specifically, the present invention includes polypeptides having the following general formula.

[R]−[D];

wherein,
[R] represents a cell-membrane permeable substance; [D] represents a fragment sequence containing Val Ile Val Ile Thr/SEQ ID NO: 27, (for example, an amino acid sequence in which Asp Ile le Ile Thr at positions 37 to 41 of the amino acid sequence set forth in SEQ ID NO: 2 (C1958) is replaced with Val Ile Val Ile Thr/SEQ ID NO: 27). In the above-described general formula, [R] and [D] can be linked directly or indirectly through a linker. Peptides, compounds having-multiple functional groups, or such can be used as a linker. Specifically, amino acid sequences containing -GGG- can be used as a linker. Alternatively, a cell-membrane permeable substance and a polypeptide containing a selected sequence can be bound to the surface of a minute particle. [R] can be linked to any positions of [D]. Specifically, [R] can be linked to the N terminal or C terminal of [D], or to a side chain of amino acids constituting [D]. Furthermore, more than one [R] molecule can be linked to one molecule of [D]. The [R] molecules can be introduced to different positions on the [D] molecule. Alternatively, [D] can be modified with a number of [R]s linked together.

For example, there have been reported a variety of naturally-occurring or artificially synthesized polypeptides having cell-membrane permeability (Joliot A. & Prochiantz A., Nat Cell Biol. 2004; 6: 189-96). All of these known cell-membrane permeable substances can be used for modifying polypeptides in the present invention. In the present invention, for example, any substance selected from the following group can be used as the above-described cell-permeable substance:

poly-arginine; Matsushita et al., J. Neurosci.; 21, 6000-6007 (2003)

[Tat/RKKRRQRRR] (SEQ ID NO: 12) Frankel, A. et al., Cell 55, 1189-1193 (1988).

Green, M. & Loewenstein, P. M. Cell 55, 1179-1188 (1988).

[Penetratin/RQIKIWFQNRRMKWKK] (SEQ ID NO: 13)

Derossi, D. et al., J. Biol. Chem. 269, 10444-10450 (1994).

[Buforin II/TRSSRAGLQFPVGRVHRLLRK] (SEQ ID NO: 14)

Park, C. B. et al., Proc. Natl. Acad. Sci. USA 97, 8245-8250 (2000).

[Transportan/GWTLNSAGYLLGKINLKALAALAKKIL] (SEQ ID NO: 15)

Pooga, M. et al., FASEB J. 12, 67-77 (1998)

[MAP (model amphipathic peptide)/KLALKLALKALKAALKLA] (SEQ ID NO: 16)

Oehlke, J. et al., Biochim. Biophys. Acta. 1414, 127-139 (1998).

[K-FGF/AAVALLPAVLLALLAP] (SEQ ID NO: 17)

Lin, Y. Z. et al., J. Biol. Chem. 270, 14255-14258 (1995).

[Ku70/VPMLK] (SEQ ID NO: 18)

Sawada, M. et al. Nature Cell Biol. 5, 352-357 (2003).

[Ku70/PMLKE] (SEQ ID NO: 25)

Sawada, M. et al. Nature Cell Biol. 5, 352-357 (2003).

[Prion/IANLGYWLLALFVTMWTDVGLCKKRPKP] (SEQ ID NO: 19)

Lundberg, P. et al., Biochem. Biophys. Res. Commun. 299, 85-90 (2002).

[PVEC/LLIILRRRIRKQAHAHSK] (SEQ ID NO: 20)

Elmquist, A. et al., Exp. Cell Res. 269, 237-244 (2001).

[Pep-1/KETWWETWWTEWSQPKKKRKV] (SEQ ID NO: 21)

Morris, M. C. et al., Nature Biotechnol. 19, 1173-1176 (2001).

[SyuB1/RGGRLSYSRRRFSTSTGR] (SEQ ID NO: 22)

Rousselle, C. et al. Mol. Pharmacol. 57, 679-686 (2000).

[Pep-7/SDLWEMMMVSLACQY] (SEQ ID NO: 23)

Gao, C. et al., Bioorg. Med. Chem. 10, 4057-4065 (2002).

[HN-1/TSPLNIHNGQKL] (SEQ ID NO: 24)

Hong, F. D. & Clayman, G. L. Cancer Res. 60, 6551-6556 (2000).

In the present invention, the poly-arginine, which is listed above as an example of cell-membrane permeable substances, is constituted by any number of arginine residues. Specifically, for example, it is constituted by consecutive 5-20 arginine residues. The preferable number of arginine residues is 11 (SEQ ID NO: 11).

### Pharmaceutical Composition Comprising VIVIT Polypeptides:

The polypeptides of the present invention inhibit proliferation of cancer cells Therefore, the present invention provides therapeutic and/or preventive agents for cancer which comprise as an active ingredient a polypeptide which comprises Val Ile Val Ile Thr/SEQ ID NO: 27 (for example, an amino acid sequence in which Asp Ile Ile Ile Thr at positions 37 to 41 of the amino acid sequence set forth in SEQ ID NO: 2 (C1958) is replaced with Val Ile Val He Thr/SEQ ID NO: 27); or a polynucleotide encoding the same. Alternatively, the present invention relates to methods for treating and/or preventing cancer comprising the step of administering a polypeptide of the present invention. Furthermore, the present invention relates to the use of the polypeptides of the present invention in manufacturing pharmaceutical compositions for treating and/or preventing cancer. Cancers which can be treated or prevented by the present invention are not limited, so long as expression of C1958 is up-regulated in the cancer cells. For example, the polypeptides of the present invention are useful for treating pancreatic cancer, lung cancer, kidney cancer or testicular tumors. Among them, pancreatic cancer is particularly preferable as a target for treatment or prevention in the present invention.

Alternatively, the polypeptides of the present invention can be used to induce apoptosis of cancer cells. Therefore, the present invention provides apoptosis inducing agents for cells, which comprise as an active ingredient a polypeptide which comprises Val Ile Val Ile Thr/SEQ ID NO: 27 (for example, an amino acid sequence in which Asp Ile Ile Ile Thr at positions 37 to 41 of the amino acid sequence set forth in SEQ ID NO: 2 (C1958) is replaced with Val Ile Val Ile Thr/SEQ ID NO: 27); or a polynucleotide encoding the same. The apoptosis inducing agents of the present invention may be used for treating cell proliferative diseases such as cancer. Cancers which can be treated or prevented by the present invention are not limited, so long as expression of C1958 is up-regulated in the cancer cells. For example, the polypeptides of the present invention are useful in treating pancreatic cancer, lung cancer, kidney cancer or testicular tumors. Among them, pancreatic cancer is particularly preferable as a target for treatment or prevention in the present invention. Alternatively, the present invention relates to methods for inducing apoptosis of cells which comprise the step of administering the polypeptides of the present invention: Furthermore, the present invention relates to the use of polypeptides of the present invention in manufacturing pharmaceutical compositions for inducing apoptosis in cells.

The polypeptides of the present invention induce apoptosis in C1958-expressing cells such as pancreatic cancer. In the meantime, C1958 expression has not been observed in most of normal organs. In some normal organs, the expression level of C1958 is relatively low as compared with cancer tissues. Accordingly, the polypeptides of the present invention may induce apoptosis specifically in cancer cells.

When the polypeptides of the present invention are administered, as a prepared pharmaceutical, to human and other mammals such as mouse, rat, guinea pig, rabbit, cat, dog, sheep, pig, cattle, monkey, baboon and chimpanzee for treating cancer or inducing apoptosis in cells, isolated compounds can be administered directly, or formulated into an appropriate dosage form using known methods for preparing pharmaceuticals. For example, if necessary, the pharmaceuticals can be orally administered as a sugar-coated tablet, capsule, elixir, and microcapsule, or alternatively parenterally administered in the injection form that is a sterilized solution or suspension with water or any other pharmaceutically acceptable liquid. For example, the compounds can be mixed with pharmacologically acceptable carriers or media, specifically sterilized water, physiological saline, plant oil, emulsifier, suspending agent, surfactant, stabilizer, corrigent, excipient, vehicle, preservative, and binder, in a unit dosage form necessary for producing a generally accepted pharmaceutical. Depending on the amount of active ingredient in these formulations, a suitable dose within the specified range can be determined.

Examples of additives that can be mixed in tablets and capsules are binders such as gelatin, corn starch, tragacanth gum, and gum arabic; media such as crystalline cellulose; swelling agents such as corn starch, gelatin, and alginic acid; lubricants such as magnesium stearate; sweetening agents such as sucrose, lactose or saccharine; and corrigents such as peppermint, wintergreen oil and cherry. When the unit dosage from is capsule, liquid carriers such as oil can be further included in the above-described ingredients. Sterilized mixture for injection can be formulated using media such as distilled water for injection according to the realization of usual pharmaceuticals.

Physiological saline, glucose, and other isotonic solutions containing adjuvants such as D-sorbitol, D-mannose, D-mannitol, and sodium chloride can be used as an aqueous solution for injection. They can be used in combination with a suitable solubilizer, for example, alcohol, specifically ethanol and polyalcohols such as propylene glycol and polyethylene glycol, non-ionic surfactants such as Polysorbate 80™ and HCO-50.

Sesame oil or soybean oil can be used as an oleaginous liquid, and also used in combination with benzyl benzoate or benzyl alcohol as a solubilizer. Furthermore, they can be further formulated with buffers such as phosphate buffer and sodium acetate buffer; analgesics such as procaine hydrochloride; stabilizers such as benzyl alcohol and phenol; and antioxidants. Injections thus prepared can be loaded into appropriate ampoules.

Methods well-known to those skilled in the art can be used for administering pharmaceutical compounds of the present invention to patients, for example, by intraarterial, intravenous, or subcutaneous injection, and similarly, by intranasal, transtracheal, intramuscular, or oral administration. Doses and administration methods are varied depending on the body weight and age of patients as well as administration methods. However, those skilled in the art can routinely select them. DNA encoding a-polypeptide of the present invention can be inserted into a vector for the gene therapy, and the vector can be administered for treatment. Although doses and administration methods are varied depending on the body weight, age, and symptoms of patients, those skilled in the art can appropriately select them. For example, a dose of the compound which bind to the polypeptides of the present invention so as to regulate their activity is, when orally administered to a normal adult (body weight 60 kg), about 0.1 mg to about 100 mg/day, preferably about 1.0 mg to about 50 mg/day, more preferably about 1.0 mg to about 20 mg/day, although it is slightly varied depending on symptoms.

When the compound is parentera

Ily administered to a normal adult (body weight 60 kg) in the injection form, it is convenient to intravenously inject a dose of about 0.01 mg to about 30 mg/day, preferably about 0.1 mg to about 20 mg/day, more preferably about 0.1 mg to about 10 mg/day, although it is slightly varied depending on patients, target organs, symptoms, and administration methods. Similarly, the compound can be administered to other animals in an amount converted from the dose for the body weight of 60 kg.

### III. Producing and Identifying Compounds to Treat Cancers

In view of the evidence provided in the examples, one aspect of the invention involves identifying test compounds that reduce or prevent the binding between C1958 and PPP3CA.

Methods for determining C1958/PPP3CA binding include any methods for determining the interaction of two proteins. Such assays include, but are not limited to, traditional approaches, such as, cross-lining, co-immunoprecipitation, and co-purification through gradients or chromatographic columns. In addition, protein-protein interactions can be monitored by using a yeast-based genetic system described by Fields and co-workers (Fields and Song, Nature 340:245-246 (1989); Chien et al., Proc. Natl. Acad. Sci. USA 88, 9578-9582 (1991)) and as disclosed by Chevray and Nathans (Proc. Natl. Acad. Sci. USA 89:5789-5793 (1992)). Many transcriptional activators, such as yeast GALA, consist of two physically discrete modular domains, one acting as the DNA-binding domain, while the other one functioning as the transcription activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GALA-activated promoter depends on reconstitution of GAL4 activity via protein-protein interaction. Colonies containing interacting polypeptides are detected with a chromogenic substrate for O-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

While the application refers to “C1958” or “PPP3CA,” it is understood that where the interaction of the two is analyzed or manipulated, it is possible to use the binding portions of one or both of the proteins in place of the full-length copies of the proteins. Fragments of C1958 that bind to PPP3CA may be readily identified using standard deletion analysis and/or mutagenesis of C1958 to identify fragments that bind to PPP3CA. Specifically, as described above, it was confirmed that C1958 polypeptide interacts with PPP3CA at PDIIIT motif thereof. Accordingly, any fragments comprising PDIIIT motif of the amino acid sequence of SEQ ID NO: 2 can be used as PPP3CA-binding fragment of C1958 polypeptide. For example, polypeptides comprising amino acid sequence from positions 36 to 41 of amino acid sequence of SEQ ID NO: 2 are conveniently used as PPP3CA-binding fragments. Furthermore, in the present invention C1958 polypeptide or PPP3CA-binding fragment of C1958 may be the phosphorylated form. The phosphorylated form of C1958 polypeptides may be prepared with a protein having C1958 kinase activity. Similar analysis may be used to identify C1958-binding fragments of PPP3CA.

As disclosed herein, any test compounds, including, e.g., proteins (including antibodies), muteins, polynucleotides, nucleic acid aptamers, and peptide and nonpeptide small organic molecules, may serve as the test compounds of the present invention. Test compounds may be isolated from natural sources, prepared synthetically or recombinantly, or any combination of the same.

For example, peptides may be produced synthetically, using solid phase techniques as described in “Solid Phase Peptide Synthesis” by G. Barany and R. B. Merrifield in Peptides, Vol. 2, edited by E. Gross and J. Meienhoffer, Academic Press, New York, N.Y., pp. 100-118 (1980). Similarly, nucleic acids can also be synthesized using the solid phase techniques, as described in Beaucage, S. L., & Iyer, R. P. (1992) Tetrahedron, 48, 2223-2311; and Matthes et al., EMBO J., 3:801-805 (1984).

Where inhibitory peptides are identified, modifications of peptides of the present invention, with various amino acid mimetics or unnatural amino acids, are particularly useful in increasing the stability of the peptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Other useful peptide modifications known in the art include glycosylation and acetylation.

Both recombinant and chemical synthesis techniques may be used to produce test compounds of the present invention. For example, a nucleic acid of test compound may be produced by insertion into an appropriate vector, which may be expressed when transfected into a competent cell. Alternatively, nucleic acids may be amplified using PCR techniques or expression in suitable hosts (see, e.g., Sambrook et al., Molecular Cloning: A Laboratory Manual, 1989, Cold Spring Harbor Laboratory, New York, USA).

Peptides and proteins may also be expressed using recombinant techniques well known in the art, e.g., by transforming suitable host cells with recombinant DNA constructs as described in Morrison, J. Bact., 132:349-351 (1977); and Clark-Curtiss & Curtiss, Methods in Enzymology, 101:347-362 (Wu et al., eds, 1983).

### Anti-C1958 and Anti-PPP3CA Antibodies

In some aspects of the present invention, test compounds are anti-C1958 or anti-PPP3CA antibodies. In some embodiments, the antibodies are chimeric, including but not limited to, humanized antibodies. In some cases, antibody embodiments of the present invention will bind either C1958 or PPP3CA at the interface where one of these proteins associates with the other. In some embodiments, these antibodies bind C1953 or PPP3CA with a Kₐ of at least about 10⁵ mol⁻¹, 10⁶ mold or greater, 10⁷ mol⁻¹, or greater, 10⁸ mol⁻¹ or greater, or 10⁹ mol⁻¹ or greater under physiological conditions. Such antibodies can be purchased from a commercial source, for example, Chemicon, Inc. (Temecula Calif.), or can be raised using as an immunogen, such as a substantially purified C1958 or PPP3CA protein, e.g., a human protein, or a fragment thereof. Methods of preparing both monoclonal and polyclonal antibodies from provided immunogens are well-known in the art. For purification techniques and methods for identifying antibodies to specific immunogens, see e.g., PCT/US02/07144 (WO/03/077838), the contents of which are incorporated by reference herein. Methods for purifying antibodies using, for example, antibody affinity matrices to form an affinity column are also well known in the art and available commercially (AntibodyShop, Copenhagen, Denmark). Identification of antibodies capable of disrupting C1958/PPP3CA association is performed using the same test assays detailed below for test compounds in general.

### Converting Enzymes

Converting enzymes may act as test compounds of the present invention. In the context of the present invention, converting enzymes are molecular catalysts that perform covalent post-translational modifications to either C1958 or PPP3CA, or both of them. Converting enzymes of the present invention will covalently modify one or more amino acid residues of C1958 and/or PPP3CA in a manner that causes either an allosteric alteration in the structure of the modified protein, or alters the C1958/PPP3CA molecular binding site chemistry or structure of the modified protein in a manner that interferes with binding between C1958 and PPP3CA. Interference with binding between the two molecules refers to a decrease in the Kₐ of binding by at least 25%, 30%, 40%, 50%, 60%, 70% or more relative to the Kₐ of binding between the proteins measured at 30° C. and an ionic strength of 0.1 in the absence of detergents. Exemplary converting enzymes of the invention include kinases, phosphatases, amidases, acetylases, glycosidase and the like.

### Constructing Test Compound Libraries

Although the construction of test compound libraries is well known in the art, the present section provides additional guidance in identifying test compounds and construction libraries of such compounds for screening for effective inhibitors of C1958/PPP3CA interaction.

### Molecular Modeling

Construction of test compound libraries is facilitated by knowledge of the molecular structure of compounds known to have the properties sought, and/or the molecular structure of the target molecules to be inhibited, i.e., C1958 and PPP3CA. One approach to preliminary screening of test compounds suitable for further evaluation is computer modeling of the interaction between the test compound and its target. In the present invention, modeling the interaction between C1958 and/or PPP3CA provides insight into both the details of the interaction itself, and suggests possible strategies for disrupting the interaction, including potential molecular inhibitors of the interaction.

Computer modeling technology allows the visualization of the three-dimensional atomic structure of a selected molecule and the rational design of new compounds that will interact with the molecule. The three-dimensional construct typically depends on data from x-ray crystallographic analysis or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modeling system described generally above consists of the CHARMm and QUANTA programs, Polygen Corporation, Walthfam, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al. Acta Pharmaceutica Fennica 97, 159-166 (1988); Ripka, New Scientist 54-57 (Jun. 16, 1988); McKinaly and Rossmann, Annu. Rev. Pharmacol. Toxicol. 29, 111-122 (1989); Perry and Davies, Prog Clin Biol Res. 291:189-93 (1989); Lewis and Dean, Proc. R. Soc. Lond. 236, 125-140 and 141-162 (1989); and, with respect to a model receptor for nucleic acid components, Askew, et al., J. Am. Chem. Soc. 111, 1082-1090 (1989).

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. See, e.g., DesJarlais et al. (1988) J. Med. Chem. 31:722; Meng et al. (1992) J. Computer Chem. 13:505; Meng et al. (1993) Proteins 17:266; Shoichet et al. (1993) Science 259:1445

Once a putative inhibitor of C1958/PPP3CA interaction has been identified, combinatorial chemistry techniques can be employed to construct any number of variants based on the chemical structure of the identified putative inhibitor, as detailed below. The resulting library of putative inhibitors, or “test compounds” may be screened using the methods of the present invention to identify test compounds of the library that disrupt C1958/PPP3CA association.

### Combinatorial Chemical Synthesis

Combinatorial libraries of test compounds may be produced as part of a rational drug design program involving knowledge of core structures existing in known inhibitors of the C1958/PPP3CA interaction. This approach allows the library to be maintained at a reasonable size, facilitating high throughput screening. Alternatively, simple, particularly short, polymeric molecular libraries may be constructed by simply synthesizing all permutations of the molecular family making up the library. An example of this latter approach would be a library of all peptides six amino acids in length. Such a peptide library could include every 6 amino acid sequence permutation. This type of library is termed a linear combinatorial chemical library.

Preparation of Combinatorial Chemical Libraries is Well Known to Those of Skill in the art, and may be generated by either chemical or biological synthesis. Combinatorial chemical libraries include, but are not limited to, peptide libraries (see, e.g., U.S. Pat. No. 5,010,175, Furka, Int. J. Pept. Prot. Res. 37:487-493 (1991) and Houghten et al, Nature 354:84-86 (1991)). Other chemistries for generating chemical diversity libraries can also be used. Such chemistries include, but are not limited to: peptides (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (DeWitt et al, Proc. Natl. Acac Sci. USA 90:6909-6913 (1993)), vinylogous polypeptides (Hagihara et al, J. Am. Chem. Soc. 114:6568 (1992)), nonpeptidal peptidomimetics with glucose scaffolding (Hirschmann et al., J. Am. Chem. Soc. 114:9217-9218 (1992)), analogous organic syntheses of small compound libraries (Chen et al, J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughan et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

### Phage Display

Another approach uses recombinant bacteriophage to produce libraries. Using the “phage method” (Scott and Smith, Science 249:386390, 1990; Cwirla, et al, Proc. Natl. Acad. Sci., 87:6378-6382, 1990; Devlin et al., Science, 249:404-406, 1990), very large libraries can be constructed (e.g., 10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghten (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### Screening Test Compound Libraries

Screening methods of the present invention provide efficient and rapid identification of test compounds that have a high probability of interfering with C1958/PPP3CA association. Generally, any method that determines the ability of a test compound to interfere with C1958/PPP3CA association is suitable for use with the present invention. For example, competitive and non-competitive inhibition assays in an ELISA format may be utilized. Control experiments should be performed to determine maximal binding capacity of system (e.g., contacting bound C1958 with PPP3CA and determining the amount of PPP3CA that binds to C1958 in the examples below).

### Competitive Assay Format

Competitive assays may be used for screening test compounds of the present invention. By way of example, a competitive ELISA format may include C1958 (or PPP3CA) bound to a solid support. The bound C1958 (or PPP3CA) would be incubated with PPP3CA (or C1958) and a test compound. After sufficient time to allow the test compound and/or PPP3CA (or C1958) to bind C1958 (or PPP3CA), the substrate would be washed to remove unbound material. The amount of PPP3CA bound to C1958 is then determined. This may be accomplished in any of a variety of ways known in the art, for example, by using an PPP3CA (or C1958) species tagged with a detectable label, or by contacting the washed substrate with a labeled anti-PPP3CA (or C1958) antibody. The amount of PPP3CA (or C1958) bound to C1958 (or PPP3CA) will be inversely proportional to the ability of the test compound to interfere with the PPP3CA/C1958 association. Protein, including but not limited to, antibody, labeling is described in Harlow & Lane, Antibodies, A Laboratory Manual (1988).

In a variation, C1958 (or PPP3CA) is labeled with an affinity tag. Labeled C-1958 (or PPP3CA) is then incubated with a test compound and PPP3CA (or C1958), then immunoprecipitated. The immunoprecipitate is then subjected to Western blotting using an anti-PPP3CA (or C1958) antibody. As with the previous competitive assay format, the amount of PPP3 CA (or C1958) found associated with C1958 (or PPP3CA) is inversely proportional to the ability of the test compound to interfere with the C1958/PPP3CA association.

### Non-Competitive Assay Format

Non-competitive binding assays may also find utility as an initial screen for testing compound libraries constructed in a format that is not readily amenable to screening using competitive assays, such as those described herein. An example of such a library is a phage display library (See, e.g., Barret, et al. (1992) Anal. Biochem 204, 357-364).

Phage libraries find utility in being able to produce quickly working quantities of large numbers of different recombinant peptides. Phage libraries do not lend themselves to competitive assays of the invention, but can be efficiently screened in a non-competitive format to determine which recombinant peptide test compounds bind C1958 or PPP3 CA. Test compounds identified as binding can then be produced and screened using a competitive assay format. Production and screening of phage and cell display libraries is well-known in the art and discussed in, for example, Ladner et al., WO 88/06630; Fuchs et al. (1991) Biotechnology 91369-1372; Goward et al. (1993) TIBS 18:136-140; Charbit et al. (1986) EMBO J. 5, 3029-3037; Cull et al. (1992) PNAS USA 89:1865-1869; Cwirla, et al. (1990) Proc. Natl. Acad. Sci. U.S.A. 87, 6378-6382.

An exemplary non-competitive assay would follow an analogous procedure to the one described for the competitive assay, without the addition of one of the components (C1958 or PPP3CA). However, as non-competitive formats determine test compound binding to C1958 or PPP3CA, the ability of test compound to bind both C1958 and PPP3CA needs to be determined for each candidate. Thus, by way of example, binding of the test compound to immobilized C1958 may be determined by washing away unbound test compound; eluting bound test compound from the support, followed by analysis of the eluate; e.g., by mass spectroscopy, protein determination (Bradford or Lowry assay, or Abs at 280 nm determination.). Alternatively, the elution step may be eliminated and binding of test compound determined by monitoring changes in the spectroscopic properties of the organic layer at the support surface. Methods for monitoring spectroscopic properties of surfaces include, but are not limited to, absorbance, reflectance, transmittance, birefringence, refractive index, diffraction, surface plasmon resonance, ellipsometry, resonant mirror techniques, grating coupled waveguide techniques and multipolar resonance spectroscopy, all of which are known to those of skill in the art. A labeled test compound may also be used in the assay to eliminate need for an elution step. In this instance, the amount of label associated with the support after washing away unbound material is directly proportional to test compound binding.

A number of well-known robotic systems have been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett Packard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St Louis, Mo., ChemStar, Ltd, Moscow, RU, 3D Pharmaceuticals, Exton, Pa., Martek Biosciences, Columbia, Md., etc.).

### Screening Converting Enzymes

Test compounds that are converting enzymes may be assayed in a noncompetitive format, using co-factors and auxiliary substrates specific for the converting enzyme being assayed. Such co-factors and auxiliary substrates are known to one of skill in the art, given the type of converting enzyme to be investigated.

One exemplary screening procedure for converting enzymes involves first contacting C1958 and/or PPP3CA with the converting enzyme in the presence of co-factors and auxiliary substrates necessary to perform covalent modification of the protein characteristic of the converting enzyme, preferably under physiologic conditions. The modified protein(s) is then tested for its ability to bind to its binding partner (i.e., binding of C1958 to PPP3CA). Binding of the modified protein to its binding partner is then compared to binding of unmodified control pairs to determine if the requisite change in Kₐ noted above has been achieved.

To facilitate detection of proteins in performing the assay, one or more proteins may be labeled with a detectable label as described above, using techniques well known to those of skill in the art.

### Methods for Screens

The screening embodiments described above are suitable for high through-put determination of test compounds suitable for further investigation. In particular, the screening of the present invention preferably comprise step of detecting an association between C1958 and PPP3CA.

Alternatively, the test compound under investigation may be added to proliferating cells and proliferation of the treated cells monitored relative to proliferation of a control population not supplemented with the test compound. Cell lines suitable for screening test compounds will be obvious to one of skill in the art provided with the teachings presented herein.

For in vivo testing, the test compound may be administered to an accepted animal model. For example, as described bellow, cell-permeable inhibitory peptide of the present invention may suppress cell growth.

### IV. Formulating Medicaments from Identified Test Compounds

Accordingly, the present invention includes medicaments and methods useful in preventing or treating cancers. These medicaments and methods comprise at least one test compound of the present invention identified as disruptive to the C1958/PPP3CA interaction in an amount effective to achieve attenuation or arrest of disease cell proliferation. More specifically, in the context of the present invention, a therapeutically effective amount means an amount effective to prevent development of, or to alleviate existing symptoms of, the subject being treated.

Individuals to be treated with methods of the present invention include any individual afflicted with cancer, including, e.g., pancreatic cancer. Such an individual can be, for example, a vertebrate such as a mammal, including a human, dog, cat, horse, cow, or goat; or any other animal, particularly a commercially important animal or a domesticated animal. For purposes of the present invention, elevated expression of marker proteins refers to a mean cellular marker protein concentration for one or both marker proteins that is at least 10%, preferably 15%, 20%, 25%, 30%, 35%, 40%, 45%, 50%, 55% or more above normal mean cellular concentration of the marker protein(s).

### Determining Therapeutic Dose Range

Determination of an effective dose range for the medicaments of the present invention is well within the capability of those skilled in the art, especially in light of the detailed disclosure provided herein. The therapeutically effective dose of a test compound can be estimated initially from cell culture assays and/or animal models. For example, a dose can be formulated in animal models to achieve a circulating concentration range that includes the IC₅₀ (the dose where 50% of the cells show the desired effects) as determined in cell culture. Toxicity and therapeutic efficacy of test compounds also can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index (i.e., the ratio between LD₅₀ and ED₅₀). Compounds which exhibit high therapeutic indices are preferable. The data obtained from these cell culture assays and animal studies may be used in formulating a dosage range for use in humans. The dosage of such compounds may Ile within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl et al., 1975, in “The Pharmacological Basis of Therapeutics”, Ch. 1 p 1. Dosage amount and interval may be adjusted individually to provide plasma levels of the active test compound sufficient to maintain the desired effects.

### Pharmaceutically Acceptable Excipients

Medicaments administered to a mammal (e.g., a human) may contain a pharmaceutically-acceptable excipient, or carrier. Suitable excipients and their formulations are described in Remington's Pharmaceutical Sciences, 16th ed., 1980, Mack Publishing Co., edited by Oslo et al. For aqueous preparations, an appropriate amount of a pharmaceutically-acceptable salt is typically used in the formulation to render the formulation isotonic. Examples of the pharmaceutically-acceptable isotonic excipients include, but are not limited to, liquids such as saline, Ringer's solution, Hanks's solution and dextrose solution. Isotonic excipients are particularly important for injectable formulations.

For transmucosal administration, penetrants appropriate to the barrier to be permeated are used in the formulation. Such penetrants are generally known in the art.

Excipients may be used to maintain the correct pH of the formulation. For optimal shelf life, the pH of solutions containing test compounds is preferably from about 5 to about 8, and more preferably from about 7 to about 7.5. The formulation may also comprise a lyophilized powder or other optional excipients suitable to the present invention including sustained release preparations such as semi-permeable matrices of solid hydrophobic polymers, which matrices are in the form of shaped articles, e.g., films, liposomes or microparticles. It will be apparent to those persons skilled in the art that certain excipients may be more preferable depending upon, for instance, the route of administration, the concentration of test compound being administered, or whether the treatment uses a medicament that includes a protein, a nucleic acid encoding the test compound, or a cell capable of secreting a test compound as the active ingredient.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes. Proper formulation is dependent upon the route of administration chosen.

For oral administration, carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by formulating a test compound with a solid dispersible excipient, optionally grinding a resulting mixture and processing the mixture of granules after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Many of the compounds of the invention may be optionally provided as salts with pharmaceutically compatible counter-ions. Pharmaceutically compatible salts may be formed with many acids, including but not limited to hydrochloric, sulfuric, acetic, lactic, tartaric, malic, succinic, etc, depending upon the application. Salts tend to be more soluble in aqueous or other protonic solvents that are the corresponding free base forms.

In addition to acceptable excipients, formulations of the present invention may include therapeutic agents other than identified test compounds. For example formulations may include anti-inflammatory agents, pain killers, chemotherapeutics, mucolytics (e.g. n-acetyl-cysteine) and the like. In addition to including other therapeutic agents in the medicament itself the medicaments of the present invention may also be administered sequentially or concurrently with the one or more other pharmacologic agents. The amounts of medicament and pharmacologic agent depend, for example, on what type of pharmacologic agent(s) is are used, the disease being treated, and the scheduling and routes of administration.

Following administration of a medicament of the invention, the mammal's physiological condition can be monitored in various ways well known to the skilled practitioner.

### Gene Therapy

Protein and peptide test compounds identified as disruptors of C1958/PPP3CA association may be therapeutically delivered using gene therapy to patients suffering from cancers. Exemplary test compounds amenable to gene therapy techniques include converting enzymes as well as peptides that directly alter the C1958/PPP3CA association by steric or allosteric interference. Alternatively, VIVIT polypeptides of the present invention can also be used as the peptides that directly alter the C1958/PPP3CA association. In some aspects, gene therapy embodiments include a nucleic acid sequence encoding a suitable identified test compound of the invention. In preferred embodiments, the nucleic acid sequence includes regulatory elements necessary for expression of the test compound in a target cell. The nucleic acid may be equipped to stably insert into the genome of the target cell (see e.g., Thomas, K. R— and Capecchi, M. R. (1987) Cell 51:503 for a description of homologous recombination cassettes vectors).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

For general reviews of the methods of gene therapy, see Goldspiel et al., (1993) Clinical Pharmacy 12:488-505; Wu and Wu, (1991) Biotherapy 3:87-95; Tolstoshev, (1993) Ann. Rev. Pharmacol Toxicol. 33:573-596; Mulligan, (1993) Science 260:926-932; and Morgan and Anderson, (1993) Ann. Rev. Biochem. 62:191-217; May, (1993) TIBTECH 11(5):155-215. Methods commonly known in the art of recombinant DNA technology which can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

### V. Screening and Treatment Kits

In one embodiment, the present invention provides an article of manufacture or kit for screening for a compound useful in treating or preventing cancers, wherein the kit comprises: (a) a PPP3CA-binding domain of a C1958 polypeptide; (b) a C1958-binding domain of a PPP3 CA polypeptide, and (c) a reagent that detects the interaction between the two polypeptides. As discussed above, the polypeptide comprising the PPP3CA-binding domain may comprise a full length C1958 polypeptide or a PPP3CA-binding portion thereof. Likewise, the polypeptide comprising the C1958-binding domain may comprise a full-length PPP3 CA polypeptide or a C1958-binding portion thereof.

The reagent that detects the interaction between the two polypeptides preferably detects an association between the polypeptide comprising the PPP3CA-binding domain and the polypeptide comprising the C1958 binding domain.

In a further embodiment of the invention, articles of manufacture and kits containing materials useful for treating the pathological conditions described herein are provided. The article of manufacture may comprise a container of a medicament as described herein with a label. Suitable containers include, for example, bottles, vials, and test tubes. The containers may be formed from a variety of materials, such as glass or plastic. In the context of the present invention, the container holds a composition having an active agent which is effective for treating a cell proliferative disease, for example, cancers. In one embodiment, the active agent in the composition is an identified test compound (e.g., antibody, small molecule, etc.) capable of disrupting C1958/PPP3CA association in vivo. The label on the container should indicate that the composition is used for treating one or more conditions characterized by abnormal cell proliferation. The label may also indicate directions for administration and monitoring techniques, such as those described herein.

In addition to the container described above, a kit of the invention may optionally comprise a second container housing a pharmaceutically-acceptable diluent. It may further include other materials desirable from a commercial and user standpoint, including other buffers, diluents, filters, needles, syringes, and package inserts with instructions for use.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

Hereinafter, the present invention is described in more detail by reference to the Examples. However, the following materials, methods and examples only illustrate aspects of the invention and in no way are intended to limit the scope of the present invention. As such, methods and materials similar or equivalent to those described herein can be used in the practice or testing of the present invention.

### EXAMPLES

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of non-critical parameters that could be changed or modified to yield essentially similar results.

### Materials and Methods

### Cell Lines and Clinical Materials

Human-pancreatic cancer cell lines, Capan-1, Capan-2, Panc-1, Aspc-1, MIApaca-2, KLM-1, PK-1, and PK59, were kindly provided by Dr. Jae-Gahb Park (Korean Cell Line Bank, Cancer Research Institute, Seoul National University College of Medicine, Korea). All cells were cultured in appropriate media: i.e. RPMI-1640 (Sigma, St. Louis, Mo.) for Capan-1, Capan-2, Panc-1, PK-1, and Aspc-1; Dulbecco's modified Eagle's medium (Invitrogen, Carlsbad, Calif.) for normal human dermal fibroblasts (NHDF), MIApaca-2, HEK293T, and Cos-7. Each medium was supplemented with 10% fetal bovine serum (Cansera) and 1% antibiotic/antimycotic solution (Sigma). Cells were maintained at 37° C. in an atmosphere of humidified air with 5% CO₂. Clinical samples (pancreatic cancer and normal pancreatic duct) were obtained from surgical specimens, concerning which all patients had given informed consent.

### Construction of Expression Vector

The entire coding sequence of C1958V1 cDNA was amplified by RT-PCR with primers, C1958V1-forward (5′-CCGGAATFCGACATGGGGCTTAAGATGTCC-3′ (SEQ ID NO.5)) and C1958V1-reverse (5′-CCGCTCGAGGGCTTCTGGGTCGATTTCTCC-3′ (SEQ ID NO.6)). The product was inserted into the EcoRI and XhoI sites of pcDNA3.1(+).myc.his (Invitrogen) or pCAGGS expression vectors.

### Immunoprecipitation and Western Blot Analysis

Cos-7 and HEK293T cells were transfected transiently with the expression vectors using FuGENE 6 (Roche) according to the manufacturer's instructions. The transfected Cos-7 cell and other pancreatic cancer cells were washed with PBS and harvested with RIPA buffer (150 mM NaCl, 1% NP-40, 50 mM Tris-HCl (pH.8.0), 0.1% SDS, 0.5% sodium deoxycholate, and 1X Protease Inhibitor Cocktail SetIII (Calbiochem)). The supernatants were standardized for protein concentration by DC protein assay (Bio-Rad). Immunoprecipitation were done with rat anti-HA antibody (Roche) and the antibodies were collected by protein G sepharose (Zymed). Proteins were separated by 10-20% gradient SDS-PAGE and immunoblotted with mouse anti-myc (Santa Cruz), anti-Flag (Sigma), anti-HA and rabbit anti-C1958 (immunized with full-length recombinant C1958 protein) antibodies.

### Immunochemical Staining

PK-1 and KLM-1 cells were fixed with PBS containing 4% paraformaldehyde for 20 min at 4° C. and permealized with PBS containing 0.1% Triton X-100 for 2.5 min at room temperature. The cells were blocked with 3% BSA in PBS for 1 h and then incubated with rabbit anti-C1958 antibody for 1 h at room temperature, followed by incubation with Alexa488-conjugated secondary antibody. Nuclei were counter-stained with 4′,6-diamidino-2-phenylindole, dihydrochloride (DAPI). Fluorescent images were obtained by confocal microscopy (Leica).

Paraffin embedded sections were treated with xylene, then the antigen was retrieved by microwave in antigen-retrieval buffer (DAKO). Endogenous peroxidase activity was blocked by incubation with Peroxidase Blocking Reagent (DAKO). The sections were blocked with Protein Block Serum-Free (DAKO) for 30 min and then incubated with the anti-C1958 antibody for 30 min at room temperature. After washing with PBS, the sections were incubated with HRP-conjugated anti-rabbit IgG (DAKO) and color developed with DAB. Finally the sections were counter-stained by hematoxylin. Images were obtained by CCD camera attached to microscopy (Olympus).

### TAP System

For construct of TAP (Tandem affinity purification) expression vector, cDNA for the TAP tag sequence, consisting of immunoglobulin G-binding domain and calmodulin-binding peptide separated with the cleavage site of Tobacco etch virus protease (TEV) with SalI at 3′ end, was PCR-amplified. Firstly, TAP tag was cloned into pcDNA-3.1(+)-myc-His expression vector. Next, pcDNA-3.1(+)-myc-His-TAP was digested with XhoI and Sail and resulted myc-His-TAP fragment was inserted into pCAGGS/neo vector. C1958V10RF cDNA was subcloned into pCAGGS-myc-His-TAP/neo expression vector. TAP-system purification was performed as described previously. Briefly, pCAGGS/neo-C1958V1-TAP or pCAGGS/neo-TAP (MOCK) as a control was transfected to Panc-1 cells. 72 hours after transfection, cells were lysed with IPP buffer (10 mM Tris-HCl (pH8.0), 0.1% NP-40, 150 mM NaCl, 1 mM NaF, containing the Protease Inhibitor Cocktail). Supernatant fraction was incubated with IgG-sepharose (Amersham Biosciences). The bound protein was incubated with TEV protease (Invitrogen) at 4° C. for overnight and eluted protein was further incubated with Calmodulin Affinity resin (Stratagene) with 1 mM CaCl₂. Finally, bound protein was eluted with 1 mM EGTA and subjected into 12% SDS-PAGE. Proteins were visualized by silver staining using Silver Stain “Daiichi” (Daiichi Pure Chemicals). Differential protein bands to the control TAP were excised from the gel and PMF-MS was custom-operated by Aproscience Co. (Tokushima, Japan).

### Cell-Growth Assays for Evaluation of Inhibitory Peptides

PK-1, Capan-1, Panc-1, NHDF, and HEK293T cells were treated with peptides (Sigma) in next day (denoted as day 0) of cell-passage. MTT assays were performed to quantify cell viability. At day 2 or subsequent days, MTT solution (3-(4,5-dimethylthiazol-2-yl)-2,5-diphenyl tetrazolium bromide) (Sigma) was added at a concentration of 0.5 mg/ml. Following incubation at 37° C. for 4 h, acid-SDS (0.01N HCl/10% SDS) was added; the suspension was mixed vigorously and then incubated overnight at 37° C. to dissolve the dark blue crystals. Absorbance at 570 nm was measured with a Microplate Reader 550 (BioRad).

Treatment of Pancreatic Cancer Xenografts with Cell Permeable Peptides

In vivo experiments were performed in our animal facility in accordance with institutional guidelines. A 0.1-ml aliquot of suspended PK-1 cells (5×10⁶ cells) was injected subcutaneously into the flanks of six-week-old female athymic mice (BALB/cA Jcl-nu). Tumor volumes were determined using the formula: 0.52×(larger diameter)×(smaller diameter)×(depth). When the xenograft reached to 100 mm³ in size, animals were randomly divided into two or three groups and received intratumoral or intravenous injection of 180 μg of 11R-C1958VIVIT, 11R-VEET, or PBS for 21 consecutive days. Tumor growth was assessed by calculating the ratio of tumor volume on the indicated day to the volume calculated on the initiation of treatment.

### Flow Cytometric Analysis

PK-1 cell was maintained as described before (RPMI with 10% FBS). The cells were incubated with or without negative control (40 μM as a final concentration) or C1958VIVIT (10, 20, and 40 μM) peptide for 12 hr. After the incubation, cells were detached by trypsin and collected to tubes and washed with PBS for 3 times. Then the cells were fixed with 67% ethanol for 30 min at room temperature (RT), washed with PBS once, and treated with RNase (2 mg/ml in PBS) for 30 min at RT. Finally the cell nuclei were stained with propidium iodide (PI) for 30 min at RT. Flow cytometric analysis was done with FACS calibur (BD).

### Results

### Expression of C1958 Protein in Pancreatic Cancer Cells and Tissue Sections.

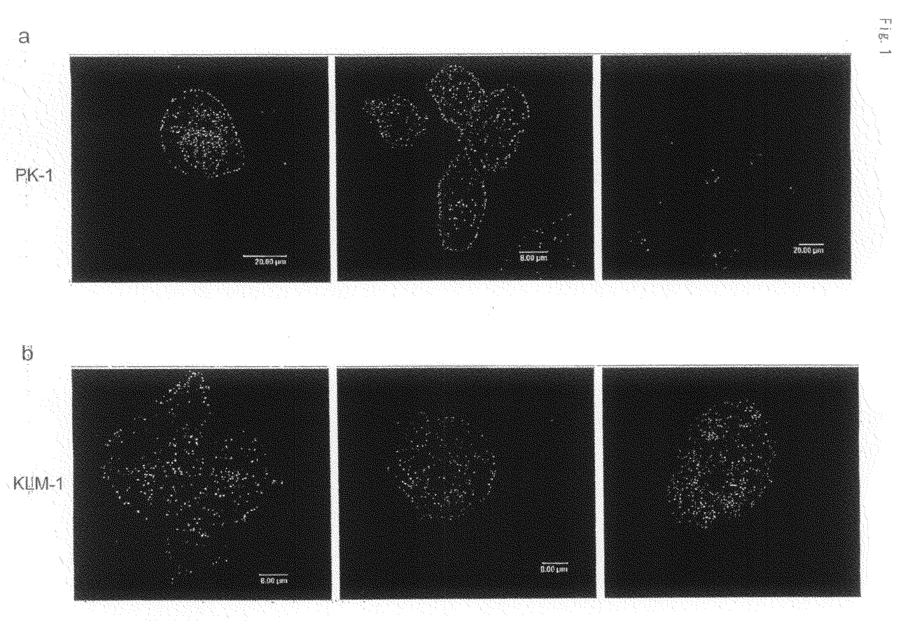

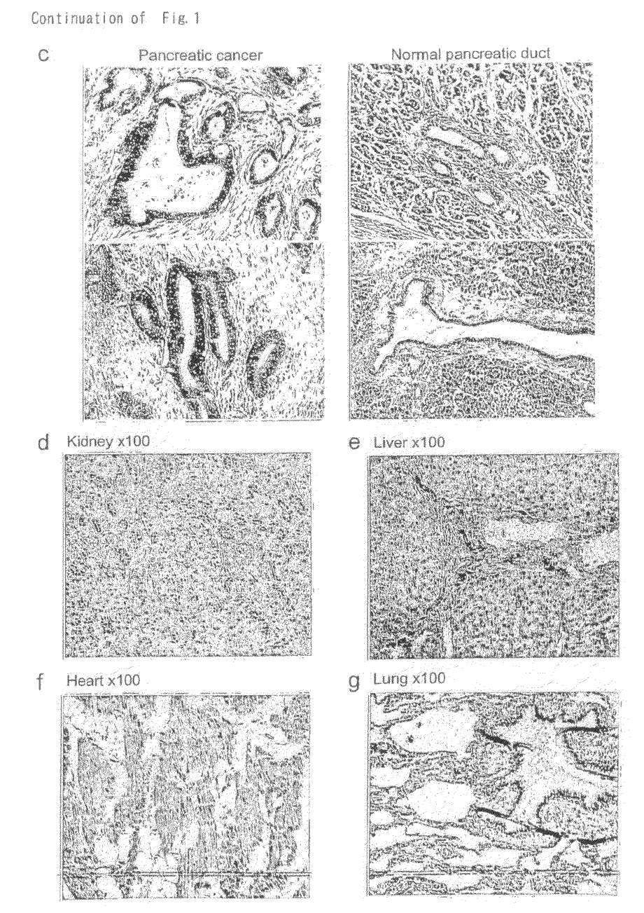

To investigate the sub-cellular localization of C1958 protein, expression of endogenous C1958 in pancreatic cancer cell lines, PK-1 and KLM-1 cells (FIG. 1a, 1b) were observed. Immunocytochemical analysis using anti-C1958 polyclonal antibody revealed that endogenous C1958 was located under the plasma membrane in both cells. Immunohistochemical analysis of C1958 using pancreatic cancer tissues and various normal human tissue sections (pancreatic duct, heart, liver, lung, and kidney) was performed. Strong staining for C1958 protein was observed in pancreatic cancer tissues, but not in normal pancreatic duct cells (FIG. 1c), as expected from the results of northern blot analysis. Staining for C1958 in the other normal tissues examined was also not or hardly detectable (FIG. 1d-1g).

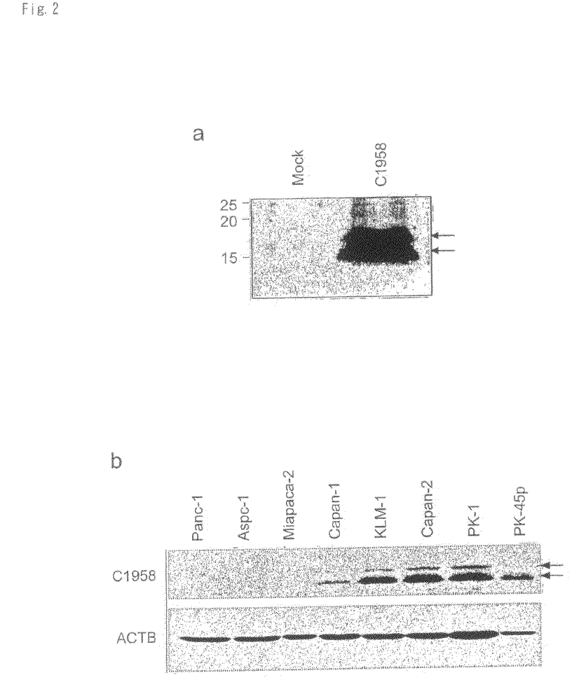

In a western blot analysis for exogenously expressed C1958 in COS-7 cells, clearly 2 bands were observed (FIG. 2a). Two different molecular weight types of C1958 were confirmed by western blot using anti-C1958 antibody in pancreatic cancer cells (FIG. 2b). The larger C1958 protein was turned to be a phosphorylated form, since the upper band was disappeared when the cell extract was treated with lambda phosphatase (data not shown). Subsequently, to determine the phosphorylation site, we transfected C1958 plasmid into KLM-1 cells and immunoprecipitated with polyclonal C1958 antibody. Mass-spectrometry analysis revealed that Thr⁴⁴ on C1958 protein is phosphorylated (data not shown).

Interaction of C1958 with PPP3CA.

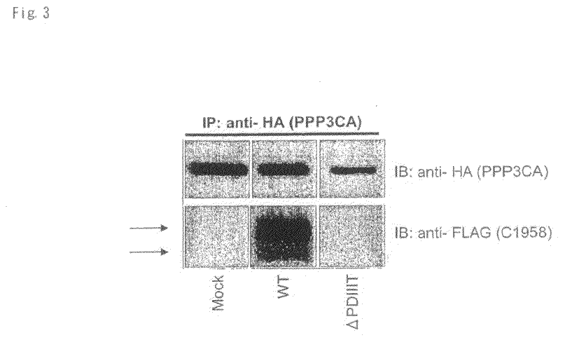

To elucidate the functional mechanism of C1958 protein in pancreatic cancer cell growth, we searched for proteins interacting with C1958 using the TAP (tandem affinity purification) system (see Materials and Methods) combined with the mass-spectrometry analysis, and identified PPP3CA (calcineurin A subunit, PP2B) as a candidate molecule to interact with C1958. We confirmed the interaction of these proteins by immunoprecipitation assay, utilizing exogenously expressed Flag-tagged C1958 and HA-tagged PPP3CA in COS7 cells (FIG. 3). We thus found that PPP3CA preferentially bounds to the phosphorylated form of C1958.

It has been known that PPP3CA also binds to the nuclear factor of activated T-cells (NFAT) and the interaction is important for the proliferation of T-cells. PPP3CA interacts with NFATs through a conserved unique sequence motif, PxIxIT (Kiani A. et al., Immunity 2000; 12(4):359-72, as review), and synthetic peptides corresponding to this region were shown to be effective inhibitors for PPP3 CA/NFAT interaction (Aramburu J. et al., Science 1999; 285:2129-33). Since C1958 also has this conserved sequence (PDIIIT), we examined whether C1958 interacts with PPP3CA through the motif. We co-transfected into COS7 cells the HA-tagged PPP3CA with Flag-tagged ΔPDIIIT-C1958 construct, in which PDIIIT sequence is deleted, and performed immunoprecipitation experiment. This results demonstrated that ΔPDIIIT-C1958 did not bound to PPP3CA, while wild-type C1958 did (FIG. 3), suggesting that the PDIIIT motif of the C1958 is essential for the interaction with PPP3CA.

Inhibition of the Interaction of C1958 and PPP3CA with Specific Peptides

To investigate the importance of the interaction between C1958 and PPP3CA in growth of pancreatic cancer cells, we designed a cell-permeable peptide, in which the PxIxIT motif of C1958 is fused to C-terminal end of eleven arginine residues (11R), to target the docking site of the interaction and interfere of it. The 11R sequence was shown to facilitate uptake of peptide into mammalian cells with high efficiency. Recently, in NFAT/PPP3CA interaction, VIVIT sequence was found to have most effective inhibition activity of the interaction among PxIxIT sequences (Aramburu J. et al, Science 1999; 285:2129-33). Therefore, in addition to a peptide corresponding to C1958-PxIxIT motif, we designed the modified C1958 peptide, in which PDIIIT was replaced by VIVIT (11R-C1958VIVIT). As a negative control, we designed a peptide 11R-C1958VEET that has VEET sequence at the docking site. The sequences of the synthesized peptides including control peptides used in the experiments are shown in Table 1.

TABLE 1

| | | SEQ |
|---|---|---|
| | peptides sequence | ID NO. |
| VIVIT | RRRRRRRRRRR-GGG-MAGPHPVIVITGPHEE | 7 |
| C1958 | RRRRRRRRRRR-GGG-KHLDVPDIIITPPTPT | 8 |
| C1958VIVIT | RRRRRRRRRRR-GGG-KHLDVPVIVITPPTPT | 9 |
| VEET | RRRRRRRRRRR-GGG-MAGPPHIVEETGPHVI | 10 |

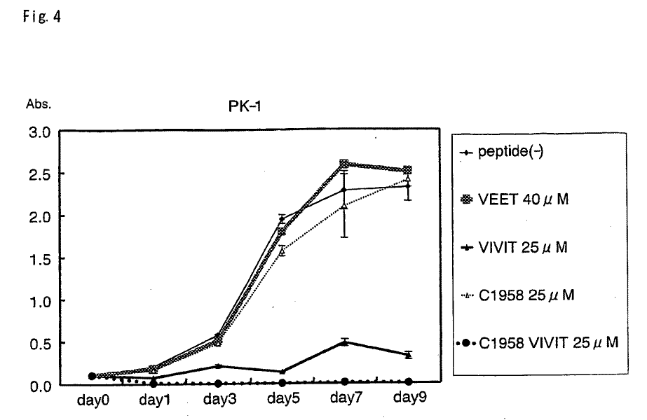

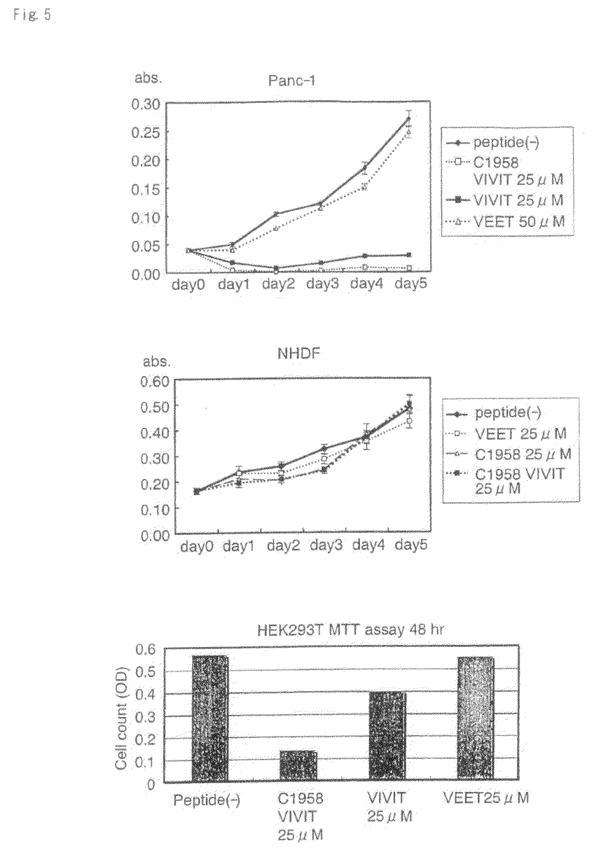

These designed peptides for inhibitory effects on the proliferation of pancreatic cancer cells were examined. PK-1 cells with 11R-C1958, 11R-C1958VIVIT, 11R-VEET, or 11R-VIVIT peptides were treated with these peptides, and performed MTT assays (FIG. 4). 11R—C1958VIVIT peptide (25 μm), but neither 11R—C1958 (25 μM) nor 11R-VEET (40 μM) peptides, significantly inhibited the growth of the cells. 11R—C1958VIVIT was more effective on growth suppression than the original VIVIT peptide at 25 μm. Similar results were obtained in Capan-1 and KLM-1 cells (data not shown). However, in control experiments, using Panc-1, HEK293T, and normal human dermal fibroblasts (NHDF) cell lines those have no or weak expression of C1958, C1958VIVIT peptide (25 μM) unexpectedly suppressed cell growth of Panc-1 and HEK293T cell lines, although almost no suppression was observed in NHDF cells (FIG. 5), suggesting a possibility that C1958VIVIT peptide also have C1958-independent growth suppression activity in some circumstances. In vitro experiments accordantly implied multiple targets in the activity of C1958VIVIT peptide that the peptide inhibited the binding between not only C1958 and PPP3CA but also NFAT and PPP3CA in immunoprecipitation assay (data not shown). Thus the peptide may also affect PPP3CA/NFAT proliferation pathway other than PPP3CA/C1958 in pancreatic cancer cells.

### C1958 VIVIT Peptide Suppress Tumor Growth In Vivo

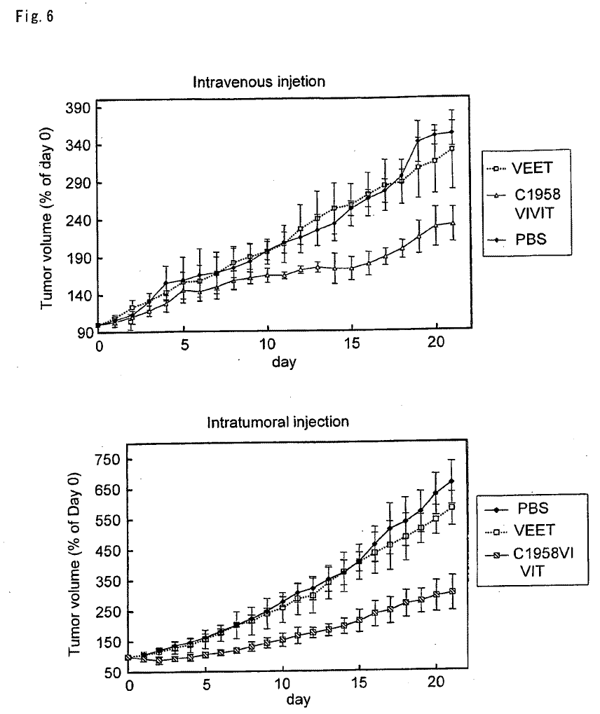

We subsequently investigated the in vivo growth inhibitory effect of 11R-C1958VIVIT peptide using mouse subcutaneous xenograft model. We injected the peptide of 9 mg/kg/day into tumor locally or intravenously for 21 consecutive days. The growth of pancreatic cancer xenografts (PK-1 cell) was significantly attenuated by the treatment with 11R-C1958VIVIT peptide, as compared to the treatment with 11R-VEET control peptide or PBS in both cases (FIG. 6), indicating that C1958VIVIT peptide has anti-tumor growth activity in vivo. In these 21 consecutive treatment days, neither body weight changes nor apparent negative effects on mice conditions were observed (data not shown), thus the peptide was proved to be a potent anti-cancer drug.

### Apoptotic Cell Death Induced by C1958-VIVIT

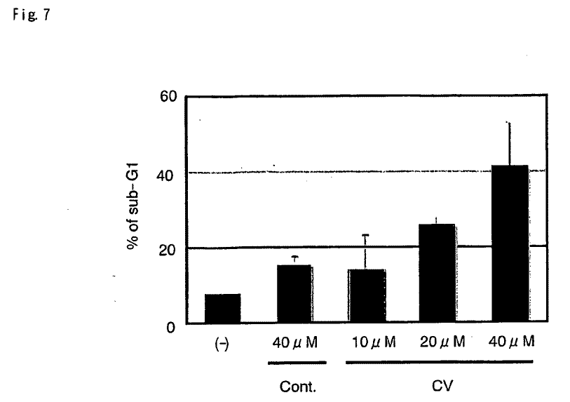

To reveal the mechanism of decreased cell growth of pancreatic cancer cells by C1958VIVIT, we performed a flow cytometric analysis and examined apoptotic cell death induction. As shown in FIG. 7, C1958VIVIT increased the sub-G1 fraction of the treated cells in a dose dependent manner, while the control peptide did not affect even at 40 μM. The result suggests that C1958VIVIT induced apoptotic cell death, as a result inhibited the cell growth.

### INDUSTRIAL APPLICABILITY

The present inventors have shown that C1958 interacts with PPP3CA, and the inhibition of the interaction leads to inhibition of cell proliferation of cancer cells. Thus, agents that inhibit the binding between C1958 and PPP3CA and prevent its activity have therapeutic utility as anti-cancer agents.

The present invention thus provides novel polypeptides and other compounds useful in treating or preventing cancer. The polypeptides of the present invention are composed of an amino acid sequence which contains VIVIT, for example a polypeptide having an amino acid sequence in which the motif sequence PxIxIT at positions 37 to 41 of the amino acid sequence set forth in SEQ ID NO: 2 (C1958V1 protein) is replaced with PVIVIT. The polypeptides of the present invention can be administered to inhibit the proliferation of, or to induce apoptosis in, cancer cells. The polypeptides of the present invention are expected to exhibit cell proliferation inhibiting effects against various cancers. Particularly, the polypeptides of the present invention have been confirmed to have cell proliferation inhibiting effects on pancreatic cancer. Pancreatic cancer is an important cancer for which an effective treatment method is still desired to be provided. Therefore, the present invention is significant in that it also provides an effective method for treating and/or preventing pancreatic cancer.

In the present invention, the treatment or preventive effect against cancer is achieved, for example, by administration of a polypeptide composed of a short amino acid sequence. The short polypeptides of the present invention can be easily and inexpensively synthesized in large scale. Furthermore, when a transfection agent is used, the treatment of cancer such as pancreatic cancer can be achieved by administering the polypeptides of the present invention into blood. Thus, the polypeptides of the present invention can be used to easily realize all the steps from manufacturing to administering, and further to delivering drug into the affected area.

Furthermore, the polypeptides merely produce amino acids even when they are decomposed in blood. This means small risk of side effects due to the degradation products of polypeptides.

All publications, databases, Genbank sequences, patents, and patent applications cited herein are hereby incorporated by reference.

While the invention has been described in detail and with reference to specific embodiments thereof, it will be apparent to one skilled in the art that various changes and modifications can be made therein without departing from the spirit and scope of the invention, the metes and bounds of which are set by the appended claims.

1. An agent for treating and/or preventing cancer comprising as an active ingredient a polypeptide which comprises Val Ile Val Ile Th/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27; a polypeptide functionally equivalent to the polypeptide; or a polynucleotide encoding the polypeptide.

2. The agent of claim 1, wherein the polypeptide consists of 5 to 30 residues.

3. The agent of claim 1, wherein the polypeptide comprises the amino acid sequence KHLDVPVIVITPPTPT/SEQ ID NO: 26.

4. The agent of claim 3, wherein the polypeptide consists of the amino acid sequence KHLDVPVIVITPPTPT/SEQ ID NO: 26.

5. The agent of claim 1, wherein the active ingredient is the polypeptide and said polypeptide is modified with a cell-membrane permeable substance.

6. The agent of claim 5, wherein the polypeptide has the following general formula:
[R]−[D];
wherein [R] represents the cell-membrane permeable substance; and [D] represents the amino acid sequence of the fragment sequence which comprises Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27; or the amino acid sequence of a polypeptide functionally equivalent to the polypeptide comprising the fragment sequence.

7. The agent of claim 6, wherein the cell-membrane permeable substance is any one selected from the group consisting of:

| | |
|---|---|
| poly-arginine; | |
| Tat/RKKRRQRRR/; | SEQ ID NO: 12 |
| Penetratin/RQIKIWFQNRRMK WKK/; | SEQ ID NO: 13 |
| Buforin II/ TRSSRAGLQFPVGRVHRLLRK/; | SEQ ID NO: 14 |
| Transportan/GWTLNSAGYLLGKINLKALAALAKKIL/; | SEQ ID NO: 15 |
| MAP (model amphipathic peptide)/KLALKLALKALKAALKLA/; | SEQ ID NO: 16 |
| K-FGF/AAVALLPAVLLALLAP/; | SEQ ID NO: 17 |
| Ku70/VPMLK/ | SEQ ID NO: 18 |
| or | |
| PMLKE/; | SEQ ID NO: 25 |
| Prion/MANLGYWLLALFVTMWTDVGLCKKRPKP/; | SEQ ID NO: 19 |
| pVEC/LLIILRRRIRKQAHAHSK/; | SEQ ID NO: 20 |
| Pep-1/KETWWETWWTEWSQPKKKRKV/; | SEQ ID NO: 21 |
| SynB1/RGGRLSYSRRRFSTSTGR/; | SEQ ID NO: 22 |
| Pep-7/SDLWEMMMVSLACQY/; | SEQ ID NO: 23 |
| and | |
| HN-1/TSPLNIHNGQKL/. | SEQ ID NO: 24 |

8. The agent of claim 7, wherein the poly-arginine is Arg 11 (SEQ ID NO: 11).

9. The agent of claim 1, wherein the cancer is any one selected from the group consisting of pancreatic cancer, lung cancer, kidney cancer, and testicular tumor.

10. A method of treating and/or preventing cancer comprising the step of administering a polypeptide comprising Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27; a polypeptide functionally equivalent to the polypeptide comprising said fragment sequence; or polynucleotides encoding those polypeptides.

11. Use of a polypeptide comprising Val Ile Val Ile Thr/SEQ ID NO: 27, wherein the polypeptide comprises at least a fragment of the amino acid sequence set forth in SEQ ID NO: 2 (C1958) in which Asp Ile Ile Ile Ile at positions 37 to 41 is replaced with Val Ile Val Ile Thr/SEQ ID NO: 27; a polypeptide functionally equivalent to the polypeptide comprising said fragment sequence; or polynucleotides encoding those polypeptides in manufacturing a pharmaceutical composition for treating and/or preventing cancer.

12. A pharmaceutical composition comprising a polypeptide comprising Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27, or a polypeptide functionally equivalent to said polypeptide; and a pharmaceutically acceptable carrier.

13. A polypeptide comprising Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27; or an amino acid sequence of a polypeptide functionally equivalent to the polypeptide comprising said fragment sequence.

14. The polypeptide of claim 13, wherein the polypeptide consists of 5 to 30 residues.

15. The polypeptide of claim 13, wherein the polypeptide comprises the amino acid sequence KHLDVPVIVITPPTPT/SEQ ID NO: 26.

16. The polypeptide of claim 15, which consists of the amino acid sequence KHLDVPVIVITPPTPT/SEQ ID NO: 26.

17. The polypeptide of claim 13, which is modified with a cell-membrane permeable substance.

18. The polypeptide of claim 13, which has the following general formula:
[R]−[D];
wherein [R] represents the cell-membrane permeable substance; and [D] represents the amino acid sequence of a fragment sequence, which comprises Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27, or the amino acid sequence of a polypeptide functionally equivalent to the polypeptide comprising said fragment sequence.

19. The polypeptide of claim 18, wherein the cell-membrane permeable substance is any one selected from the group consisting of:

| | |
|---|---|
| poly-arginine; | |
| Tat/RKKRRQRRR/; | SEQ ID NO: 12 |
| Penetratin/RQIKIWFQNRRMKWKK/; | SEQ ID NO: 13 |
| Buforin II/TRSSRAGLQFPVGRVHRLLRK/; | SEQ ID NO: 14 |
| Transportan/GWTLNSAGYLLGKINLKALAALAKKIL/; | SEQ ID NO: 15 |
| MAP (model amphipathic peptide)/KLALKLALKALKAALKLA/; | SEQ ID NO: 16 |
| K-FGF/AAVALLPAVLLALLAP/; | SEQ ID NO: 17 |
| Ku70/VPMLK/ | SEQ ID NO: 18 |
| Ku70/PMLKE/; | SEQ ID NO: 25 |
| Prion/MANLGYWLLALFVTMWTDVGLCKKRPKP/; | SEQ ID NO: 19 |
| pVEC/LLIILRRRIRKQAHAHSK/; | SEQ ID NO: 20 |
| Pep-1/KETWWETWWTEWSQPKKKRKV/; | SEQ ID NO: 21 |
| SynB1/RGGRLSYSRRRFSTSTGR/; | SEQ ID NO: 22 |
| Pep-7/SDLWEMMMVSLACQY/; | SEQ ID NO: 23 |
| and | |
| HN-1/TSPLNIHNGQKL/. | SEQ ID NO: 24 |

20. The polypeptide of claim 19, wherein the poly-arginine is Arg 11 (RRRRRRRRRRR/SEQ ID NO: 11).

21. A method of screening for a compound useful in treating or preventing cancers, said method comprising the steps of:
(a) contacting a polypeptide comprising a PPP3CA-binding domain of a C1958 polypeptide with a polypeptide comprising a C1958-binding domain of a PPP3CA polypeptide in the presence of a test compound;
(b) detecting binding between the polypeptides; and
(c) selecting a test compound that inhibits binding between the polypeptides.

22. The method of claim 21, wherein the polypeptide comprising the PPP3CA-binding domain comprises a C1958 polypeptide.

23. The method of claim 21, wherein the polypeptide comprising the PPP3CA-binding domain is a polypeptide comprising the amino acid sequence from positions 36 to 41 of the amino acid sequence of SEQ ID NO: 2.

24. The method of claim 21, wherein the polypeptide comprising the C1958-binding domain comprises a PPP3CA polypeptide.

25. The method of claim 21, wherein the polypeptide comprising the PPP3CA-binding domain is expressed in a living cell.

26. The method of claim 21, wherein the binding between the polypeptides is detected by a method comprising the step of detecting an association between the polypeptide comprising the PPP3CA-binding domain and the polypeptide comprising the C1958 binding domain.

27. A kit for screening for a compound for treating or preventing cancers, wherein the kit comprises:
(a) a polypeptide comprising a PPP3CA-binding domain of a C1958 polypeptide,
(b) a polypeptide comprising a C1958-binding domain of a PPP3CA polypeptide, and
(c) a reagent to detect the interaction between the polypeptides.

28. The kit of claim 27, wherein the polypeptide comprising the PPP3CA-binding domain comprises a C1958 polypeptide.

29. The kit of claim 27, wherein the polypeptide comprising the PPP3CA-binding domain is a polypeptide comprising the amino acid sequence from positions 36 to 41 of the amino acid sequence of SEQ ID NO: 2.

30. The kit of claim 27, wherein the polypeptide comprising the C1958-binding domain comprises a PPP3CA polypeptide.

31. The kit of claim 27, wherein the polypeptide comprising the PPP3CA-binding domain is expressed in a living cell.

32. The kit of claim 27, wherein the reagent to detect the interaction between the polypeptides comprises a reagent that detects an association between the polypeptide comprising the PPP3CA-binding domain and the polypeptide comprising the C1958 binding domain.

33. A method for treating or preventing cancers in a subject, said method comprising the step of administering a pharmaceutically effective amount of the compound selected by the method of claim 21.

34. A method for treating or preventing cancers in a subject; wherein the method comprises the step of administering a pharmaceutically effective amount of a compound that inhibits the binding between a C1958 polypeptide and a PPP3CA polypeptide.

35. A composition for treating or preventing cancers, wherein the composition comprises a pharmaceutically effective amount of the compound selected by the method of claim 21, and a pharmaceutically acceptable carrier.

36. A composition for treating or preventing cancers, wherein the composition comprises a pharmaceutically effective amount of a compound that inhibits the binding between a C1958 polypeptide and a PPP3CA polypeptide, and a pharmaceutically acceptable carrier.

37. A method for inducing apoptosis in a cell, which comprises the step of introducing a polypeptide having a dominant-negative effect against C1958 or a polynucleotide encoding the polypeptide into the cell, wherein the polypeptide comprises a fragment sequence which comprises Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27; or introducing a polypeptide functionally equivalent to the polypeptide.

38. The method of claim 37, wherein the cell is selected from the group consisting of a pancreatic cancer cell, lung cancer cell, renal cancer cell, and testicular seminoma cell.

39. A composition for inducing apoptosis in a cell, which comprises a polypeptide having a dominant-negative effect against C1958 or a polynucleotide encoding the polypeptide, wherein the polypeptide comprises a fragment sequence which comprises Val Ile Val Ile Thr/SEQ ID NO: 27, wherein the polypeptide comprises at least a fragment of the amino acid sequence set forth in SEQ ID NO: 2 (C1958) in which Asp Ile Ile Ile Thr at positions 37 to 41 is replaced with Val Ile Val Ile Thr/SEQ ID NO: 27, or comprises a polypeptide functionally equivalent to the polypeptide.


SEQUENCE LISTING (27 sequences)

SEQ ID NO: 1 - DNA, 881 bp, Homo sapiens
  CDS (163)..(390)
gggccatgacccccgctgctctgtcttgcaggctcgtcgccgcggccccccgagcccgaccgccgccgccaccaccaccagcgcccgggcgggcctcgcgcgcctcgggcgcggctccgcagtgagcccaccaagaaggaagcggcctgcagaggtgccgacatggggcttaagatgtcctgcctgaaaggctttcaaatgtgtgtcagcagcagcagcagcagccacgacgaggcccccgtcctgaacgacaagcacctggacgtgcccgacatcatcatcacgccccccacccccacgggcatgatgctgccgagggacttggggagcacagtctggctggatgagacagggtcgtgcccagatgatggagaaatcgacccagaagcctgaggaggtgtcctgggtttggctggctggctcctgctccagcggcccggcttcaggtgtccgggggcgtggctgcctggagcaggtgtgctgaataccctggatgggaactgagcgaacccgggcctccgctcagagagacgtggcaggaccagcgaggaatccagcctgtccacttccagaacagtgtttcccaggccccgctgagtggaccggacctctgacacctccaggttcttgctgactccggcctggtgaaagggagcgccatggtcctggctgttggggtcccagggagaggctctcttctggacaaacacaccctcccagcccccagggctgtgcaaacacatgcccctgccataagcaccaacaagaacttcttgcaggtggagtggctgttttttataagttgttttacagatacggaaacagtccaaaatgggatttataatttcttttttgcattataaataaagatcctctgtaac

SEQ ID NO: 2 - protein, 76 aa, Homo sapiens
MGLKMSCLKGFQMCVSSSSSSHDEAPVLNDKHLDVPDIIITPPTPTGMMLPRDLGSTVWLDETGSCPDDGEIDPEA

SEQ ID NO: 3 - DNA, 893 bp, Homo sapiens
  CDS (197)..(256)
ccgcgggaggcgcgcggctgcccgagcgccggccgggccatgacccccgctgctctgtcttgcaggctcgtcgccgcggccccccgagcccgaccgccgccgccaccaccaccagcgcccgggcgggcctcgcgcgcctcgggcgcggctccgcagtgagcccaccaagaaggaagcggcctgcagaggtgccgacatggggcttaagatgtcctgcctgaaagcagcagcagcagccacgacgaggcccccgtcctgaacgacaagcacctggacgtgcccgacatcatcatcacgccccccacccccacgggcatgatgctgccgagggacttggggagcacagtctggctggatgagacagggtcgtgcccagatgatggagaaatcgacccagaagcctgaggaggtgtcctgggtttggctggctggctcctgctccagcggcccggcttcaggtgtccgggggcgtggctgcctggagcaggtgtgctgaataccctggatgggaactgagcgaacccgggcctccgctcagagagacgtggcaggaccagcgaggaatccagcctgtccacttccagaacagtgtttcccaggccccgctgagtggaccggacctctgacacctccaggttcttgctgactccggcctggtgaaagggagcgccatggtcctggctgttggggtcccagggagaggctctcttctggacaaacacaccctcccagcccccagggctgtgcaaacacatgcccctgccataagcaccaacaagaacttcttgcaggtggagtggctgttttttataagttgttttacagatacggaaacagtccaaaatgggatttataatttcttttttgcattataaataaagatcctctgtaac

SEQ ID NO: 4 - protein, 20 aa, Homo sapiens
MGLKMSCLKAAAAATTRPPS

SEQ ID NO: 5 - DNA, 30 bp, Artificial
  Artificially synthesized primer for RT-PCR
ccggaattcgacatggggcttaagatgtcc

SEQ ID NO: 6 - DNA, 30 bp, Artificial
  Artificially synthesized primer for RT-PCR
ccgctcgagggcttctgggtcgatttctcc

SEQ ID NO: 7 - protein, 30 aa, Artificial
  Artificially synthesized peptide for inhibition of the interaction of C1958 and PPP3CA as control
RRRRRRRRRRRGGGMAGPHPVIVITGPHEE

SEQ ID NO: 8 - protein, 30 aa, Artificial
  Artificially synthesized peptide for inhibition of the interaction of C1958 and PPP3CA
RRRRRRRRRRRGGGKHLDVPDIIITPPTPT

SEQ ID NO: 9 - protein, 30 aa, Artificial
  Artificially synthesized peptide for inhibition of the interaction of C1958 and PPP3CA
RRRRRRRRRRRGGGKHLDVPVIVITPPTPT

SEQ ID NO: 10 - protein, 30 aa, Artificial
  Artificially synthesized peptide for inhibition of the interaction of C1958 and PPP3CA as control
RRRRRRRRRRRGGGMAGPPHIVEETGPHVI

SEQ ID NO: 11 - protein, 11 aa, Artificial
  an artificially synthesized 11 mer poly-arginine sequence
RRRRRRRRRRR

SEQ ID NO: 12 - protein, 9 aa, Artificial
  an artificially synthesized Tat sequence
RKKRRQRRR

SEQ ID NO: 13 - protein, 16 aa, Artificial
  an artificially synthesized Penetratin sequence
RQIKIWFQNRRMKWKK

SEQ ID NO: 14 - protein, 21 aa, Artificial
  an artificially synthesized Buforin II sequence
TRSSRAGLQFPVGRVHRLLRK

SEQ ID NO: 15 - protein, 27 aa, Artificial
  an artificially synthesized Transportan sequence
GWTLNSAGYLLGKINLKALAALAKKIL

SEQ ID NO: 16 - protein, 18 aa, Artificial
  an artificially synthesized MAP (model amphipathic peptide) seque
KLALKLALKALKAALKLA

SEQ ID NO: 17 - protein, 16 aa, Artificial
  an artificially synthesized K-FGF sequence
AAVALLPAVLLALLAP

SEQ ID NO: 18 - protein, 5 aa, Artificial
  an artificially synthesized Ku70 sequence
VPMLK

SEQ ID NO: 19 - protein, 28 aa, Artificial
  an artificially synthesized Prion sequence
MANLGYWLLALFVTMWTDVGLCKKRPKP

SEQ ID NO: 20 - protein, 18 aa, Artificial
  an artificially synthesized pVEC sequence
LLIILRRRIRKQAHAHSK

SEQ ID NO: 21 - protein, 21 aa, Artificial
  an artificially synthesized Pep-1 sequence
KETWWETWWTEWSQPKKKRKV

SEQ ID NO: 22 - protein, 18 aa, Artificial
  an artificially synthesized SynB1 sequence
RGGRLSYSRRRFSTSTGR

SEQ ID NO: 23 - protein, 15 aa, Artificial
  an artificially synthesized Pep-7 sequence
SDLWEMMMVSLACQY

SEQ ID NO: 24 - protein, 12 aa, Artificial
  an artificially synthesized HN-1 sequence
TSPLNIHNGQKL

SEQ ID NO: 25 - protein, 5 aa, Artificial
  an artificially synthesized Ku70 sequence
PMLKE

SEQ ID NO: 26 - protein, 16 aa, Artificial
  an artificially synthesized peptide sequence
KHLDVPVIVITPPTPT

SEQ ID NO: 27 - protein, 5 aa, Artificial
  an artificially synthesized peptide sequence
VIVIT